(19) United States
(12) Patent Application Publication
(10) Pub. No.: US 2026/0294326 A1
(43) Pub. Date: Oct. 1, 2026

(54) A MEDICAL DEVICE SYSTEM CONFIGURED TO DETERMINE A PROGRESSION OF PARKINSON'S DISEASE BASED ON SIGNALS COLLECTED BY A MEDICAL DEVICE IMPLANTED NEAR THE BRAIN

(71) Applicant: MEDTRONIC, INC., Minneapolis, MN (US)

(72) Inventors: Randal C. Schulhauser, Phoenix, AZ (US); Richard J. O'Brien, Hugo, MN (US); Scott T. McMahan, Wheat Ridge, CO (US); Walton W. Baxter, San Clemente, CA (US); Eric J. Panken, Edina, MN (US); Philip E. Tracton, Porter Ranch, CA (US); Eric M. Christensen, Dedham, IA (US); William Valls, Chandler, AZ (US); Jill M. Wallace, Gilbert, AZ (US)

(21) Appl. No.: 19/478,720

(22) PCT Filed: Apr. 11, 2024
(86) PCT No.: PCT/IB2024/053555

§ 371 Date: Oct. 24, 2025

Related U.S. Application Data
(60) Provisional application No. 63/498,432, filed on Apr. 26, 2023.

(51) Int. Cl. A61B 5/00; A61B 5/0205; A61B 5/024; A61B 5/11; A61B 5/145; A61B 5/287; A61B 5/293; A61B 5/374

(52) U.S. Cl. CPC A61B 5/4082; A61B 5/0205; A61B 5/374; A61B 5/4842; A61B 5/686; A61B 5/725; A61B 5/02405; A61B 5/1101; A61B 5/14546; A61B 5/287; A61B 5/293; A61B 2562/0219

(57) ABSTRACT

A medical device system includes an implantable medical device comprising one or more electrodes and accelerometer circuitry, wherein the implantable medical device is configured to be located subcutaneously and proximate to a brain of a patient. The implantable medical device is configured to sense, via the one or more electrodes, a first biometric signal that indicates electrical brain activity of the patient; sense, via the one or more electrodes, a second biometric signal that indicates cardiac activity of the patient; and generate, using the accelerometer circuitry, an accelerometer signal that indicates motion activity of the patient. Additionally, the medical device system includes processing circuitry configured to: determine, based on the first biometric signal, the second biometric signal, and the accelerometer signal, a patient status that indicates a progression of Parkinson's disease over a period of time.

This Application claims priority from U.S. Provisional Patent Application 63/498,432, filed 26 Apr. 2023, the entire content of which is incorporated herein by reference.

### TECHNICAL FIELD

This disclosure is directed to medical devices and, more particularly, to systems and methods for monitoring patient conditions.

### BACKGROUND

Patient conditions, such as Parkinson's Disease, can be tracked using biometric signals. Parkinson's disease is a neurodegenerative disease that causes the progressive loss of functioning neurons in the brain. Although no cure for Parkinson's Disease has been found, the condition can be treated and symptoms can be mitigated. Since Parkinson's Disease progresses over time, clinicians track the progression of the disease to determine appropriate treatments for the patient. A medical device may sense one or more signals that indicate a progression of Parkinson's disease over a period of time. A medical device system may analyze the one or more signals to track the progression of Parkinson's disease or other movement disorders.

### SUMMARY

In general, the disclosure is directed to techniques for operating a medical device system to generate at least one of a detection, prediction, or a classification of a condition of the patient, such as Parkinson's Disease, other movement disorders, stroke, seizure, vasovagal syncope, or psychogenic attacks. In some examples, the detection, prediction, or classification is generated based on sensor signals sensed by a single sensor device disposed above the shoulders of the patient, e.g., at a rear portion of a neck or skull base of the patient. In some examples, the sensor device may be disposed submuscularly at a side of the patient's head proximate to a temporal lobe of the patient's brain. The techniques may include sensing both brain and cardiac electrical signals via electrodes of the sensor device disposed above the shoulders, determining values of brain and cardiac parameters based on the respective signals, and generating the detection, prediction, or classification based on the parameters and a motion signal from a motion sensor of the sensing device.

It may be beneficial to track Parkinson's Disease or other movement disorders using a one or more biometric signals sensed by a medical device implanted at the rear portion of the neck or skull base of the patient. Since Parkinson's Disease is a neurogenerative disorder, certain aspects of the conditions can be tracked based on an electroencephalography (EEG) signal sensed by the medical device which is implanted proximate to the patient's brain. This EEG signal may indicate brain activity of the patient, and the system may process the EEG signal to analyze the brain activity of the patient.

Parkinson's Disease also causes one or more physical symptoms, including body tremors. An accelerometer of the medical device may generate an accelerometer signal that indicates a motion of the patient. Based on the accelerometer signal, the system ay identify a presence, a frequency, and/or a severity of one or more tremors relating to Parkinson's disease. The system may also determine one or more factors based on a combination of the EEG signal and the accelerometer signal, such as daytime sleepiness and sleep quality.

The medical device may also sense a biomarker signal that is a surrogate of the patient's cardiac function. Parkinson's Disease affects the cardiac function of the patient, so it is beneficial to monitor cardiac signals to track the condition. Cardiac activity may indicate a state of a patient's Parkinson's disease or a state of one or more other movement disorders. For example, elevated heart rate variability may be more common with patients who eventually develop Parkinson's Disease or other movement disorders as compared with patients who do not develop Parkinson's Disease or other movement disorders. This means that elevated heart rate variability may represent an early non-motor symptom of Parkinson's Disease. A medical device that is implanted at the rear portion of the neck or skull base of the patient may be configured to sense one or more biometric signals that are indicative of one or more aspects of a patient's cardiac activity such as heart rate and heart rate variability.

In some examples, the medical device is configured to sense, via one or more electrodes, a biometric signal that indicates a level of α-synuclein in a tissue area of the patient proximate to the medical device. The level of α-synuclein in the tissue area of the patient may indicate one or more aspects of the patient's cardiac activity. For example, the level of α-synuclein may represent a surrogate parameter for an electrocardiogram (ECG), electrogram (EGM), or other cardiac potential signal.

The techniques of this disclosure may provide one or more advantages. For example, by generating a set of signals using a single medical device implanted near the base of the patient's skull, the system may effectively monitor Parkinson's Disease without a using a wide variety of devices and sensors. Since the medical device is implanted, it can collect data over a long period of time, which is preferable to using external devices that are easily removed to stop collecting data. Furthermore, sensing brain signals, sensing cardiac signals, and generating motion signals using a single medical device implanted at the base of the neck allows the system to more effectively track Parkinson's Disease as compared with systems that collect signals using devices at different locations on the body, because the signals are collected from the same perspective over the same period of time. This allows the system to analyze the signals to determine the progression of Parkinson's Disease over the period of time.

Additionally, the techniques and systems of this disclosure may be implemented in an implantable medical device (IMD) that can continuously (e.g., on a periodic or triggered basis without human intervention) sense the data while subcutaneously implanted in a patient over months or years and perform numerous operations per second on patient data to enable the systems herein to monitor a progression of Parkinson's Disease. Using techniques of this disclosure with an IMD may be advantageous when a physician cannot be continuously present with the patient over weeks or months to evaluate the cardiac EGM and/or where performing the operations on the cardiac EGM described herein (e.g., modification of the cardiac EGM, generation of feature sets, and application of a machine learning model) on weeks or months of EGM data could not practically be performed in the mind of a physician.

In one example, a medical device system includes: an implantable medical device comprising: one or more electrodes; and accelerometer circuitry, wherein the implantable medical device is configured to be located subcutaneously and proximate to a brain of a patient. The implantable medical device is configured to: sense, via the one or more electrodes, a first biometric signal that indicates electrical brain activity of the patient; sense, via the one or more electrodes, a second biometric signal that indicates cardiac activity of the patient; and generate, using the accelerometer circuitry, an accelerometer signal that indicates motion activity of the patient. Additionally, the medical device system includes processing circuitry configured to: determine, based on the first biometric signal, the second biometric signal, and the accelerometer signal, a patient status that indicates a progression of Parkinson's disease over a period of time.

In another example, a method of controlling operation of a medical device system comprising an implantable medical device located subcutaneously and proximate to a brain of a patient, the comprising: sensing, via one or more electrodes of the implantable medical device, a first biometric signal that indicates electrical brain activity of the patient, wherein the implantable medical device is configured to be located subcutaneously and proximate to a brain of a patient; sensing, via the one or more electrodes, a second biometric signal that indicates cardiac activity of the patient; generating, using accelerometer circuitry of the implantable medical device, an accelerometer signal that indicates motion activity of the patient; and determining, by processing circuitry of the medical device system based on the first biometric signal, the second biometric signal, and the accelerometer signal, a patient status that indicates a progression of Parkinson's disease over a period of time.

In another examples, a non-transitory computer-readable medium includes instructions for causing one or more processors to: sense, via one or more electrodes of an implantable medical device, a first biometric signal that indicates electrical brain activity of the patient, wherein the implantable medical device is configured to be located subcutaneously and proximate to a brain of a patient; sense, via the one or more electrodes, a second biometric signal that indicates cardiac activity of the patient; generate, using accelerometer circuitry of the implantable medical device, an accelerometer signal that indicates motion activity of the patient; and determining, based on the first biometric signal, the second biometric signal, and the accelerometer signal, a patient status that indicates a progression of Parkinson's disease over a period of time.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A is a conceptual diagram of a first system configured to detect a medical condition of a patient, in accordance with one or more techniques of this disclosure.

FIG. 1B is a conceptual diagram illustrating a second system configured to detect a medical condition of a patient, in accordance with one or more techniques of this disclosure.

FIG. 1C is a conceptual diagram illustrating a third system configured to detect a medical condition of a patient, in accordance with one or more techniques of this disclosure.

FIG. 1D is a diagram of a 10-20 map for electroencephalography (EEG) sensor measurements, in accordance with one or more techniques of this disclosure.

FIG. 2A is a conceptual diagram illustrating a top view of a sensor device (e.g., an IMD), in accordance with one or more techniques of this disclosure.

FIG. 2B is a conceptual diagram illustrating a side view of the sensor device shown in FIG. 2A, in accordance with one or more techniques of this disclosure.

FIG. 2C is a conceptual diagram illustrating a top view of another example sensor device, in accordance with one or more techniques of this disclosure.

FIG. 2D depicts a side view of another example sensor device, in accordance with one or more techniques of this disclosure.

FIG. 2E depicts a side view of another example sensor device, in accordance with one or more techniques of this disclosure.

FIG. 2F depicts a side view of another example sensor device, in accordance with one or more techniques of this disclosure.

FIG. 2G depicts a top view of another example sensor device, in accordance with one or more techniques of this disclosure.

FIG. 2H depicts a top view of another example sensor device, in accordance with one or more techniques of this disclosure.

FIGS. 3A-3D depict other example sensor devices, in accordance with one or more techniques of this disclosure.

FIG. 4 is a block diagram of an example configuration of a sensor device configured to sense signals used to generate at least one of a detection, prediction, or classification of a condition of a patient, in accordance with one or more techniques of this disclosure.

FIG. 5 is a block diagram of an example configuration of an external device configured to communicate with any sensor device described herein, in accordance with one or more techniques of this disclosure.

FIG. 6 is a block diagram illustrating an example system that includes an access point, a network, external computing devices, such as a server, and one or more other computing devices, which may be coupled to a sensor device, an external device, and processing circuitry via the network, in accordance with one or more techniques described herein.

FIG. 7 is a flow diagram illustrating an example operation for determining a patient status that indicates a progression of Parkinson's disease, in accordance with one or more techniques of this disclosure.

### DETAILED DESCRIPTION

This disclosure describes various systems, devices, and techniques for detecting, monitoring, predicting, and/or classifying one or more patient conditions from a device located on the head of the patient. Parkinson's Disease and other movement disorders are neurodegenerative diseases that progress over a long period of time. Clinicians may continuously update therapies delivered to a patient based on the progression of Parkinson's Disease, so it may be beneficial to track the disease to determine appropriate therapies over time.

As described herein, a sensor device system may be used to detect, predict, and/or classify patient conditions from a location on or near the head of the patient. A sensor device may be configured to be implanted subcutaneously or positioned external (e.g., worn) on the patient without the need for any medical leads. Instead of leads, the sensor device may include a housing that carries multiple electrodes directly on the housing, and one or more other sensors on or within the housing. Using the housing electrodes, the sensor device may sense electrical signals from one or more vectors, and processing circuitry may determine values physiological parameters representative of patient condition. The signals and parameters may be indicative of brain activity and/or activity of other organs such as the heart. Based on the parameter values, the processing circuitry may detect, predict, and/or classify patient conditions. The processing circuitry may output an indication of the detection, prediction, and/or classification to a computing device, e.g., to facilitate a treatment or intervention.

Conventional electroencephalogram (EEG) electrodes are typically positioned over a large portion of a user's scalp. While electrodes in this region are well positioned to detect electrical activity from the patient's brain, there are certain drawbacks. Sensors in this location interfere with patient movement and daily activities, making them impractical for prolonged monitoring. Additionally, implanting electrodes under the patient's scalp is difficult and may lead to significant patient discomfort. To address these and other shortcomings of conventional EEG sensors, sensor devices according to technology described herein sense electrical signals from a smaller region near or on the patient's head, such as adjacent a rear portion of the patient's neck or base the patient's skull or near the patient's temple. In these positions, implantation under the patient's skin is relatively simple, and a temporary application of a wearable sensor device (e.g., coupled to a bandage, garment, band, or adhesive member) does not unduly interfere with patient movement and activity.

However, the EEG signals detected via electrodes disposed at or adjacent the back of a patient's neck may include other signals and relatively high noise amplitude. For example, the electrical signals associated with brain activity may be intermixed with electrical signals associated with cardiac activity (e.g., electrocardiogram (ECG) signals) and muscle activity (e.g., electromyogram (EMG) signals) and artifacts from other electrical sources such as patient movement or external interference. Accordingly, in some examples, the signals may be filtered or otherwise manipulated to separate the brain activity data (e.g., EEG signals) and cardiac electrical signals (e.g., ECG signals) from each other and other electrical signals (e.g., EMG signals, etc.). A sensor device of this disclosure may include multiple electrodes having non-parallel vector axes for sensing differential signals, and circuitry in the device may be configured to generate an ECG signal and an EEG signal based on the differential signals.

In some examples, a cardiac activity of the patient may indicate a status of Parkinson's disease. The medical device may be configured to detect one or more electrical signals that indicate a cardiac activity of the patient. For example, electrical signals associated with cardiac activity may include one or more electrocardiogram (ECG) signals. In some examples, the medical device may filter one or more electrical signals sensed via one or more electrodes of the medical device to identify ECG signals. Additionally, or alternatively, the medical device may sense a signal via one or more electrodes of the medical device that indicates a level of α-synuclein in a tissue area of the patient proximate to the medical device at the back of the patient's neck. In some examples, the level of α-synuclein may indicate a progress of Parkinson's disease. The level of α-synuclein may, in some cases, represent a surrogate parameter for ECG, EGM, and/or other cardiac potential signals. For example, the level of α-synuclein may indicate one or more cardiac events (e.g., atrial depolarization ventricular depolarization) that cardiac potential signals also indicate. In some examples, an external device may collect a biometric signal indicating a level of α-synuclein. In some examples, an implantable medical device may collect a biometric signal indicating a level of α-synuclein.

The sensor device may, in some examples, include an accelerometer that is configured to sense an accelerometer signal indicative of a motion of the patient. This motion signal may indicate patient movements such as tremors relating to Parkinson's Disease. It may be beneficial to track the accelerometer signal to determine whether tremors are increasing in frequency and/or severity. In some examples, an accelerometer of a medical device implanted under the patient's skin near the back of a patient's neck may be configured to generate an accelerometer signal that more reliably indicates one or more patient movements associated with tremors as compared with accelerometer signals that are generated by devices that are not implanted underneath the patient's skin. Although Parkinson's tremors often occur in a patient's limbs, tremors may also occur in the patient's jaw, chin, or mouth. A medical device implanted underneath the patient's skin at the back of the patient's neck may be configured to generate an accelerometer signal that indicates one or more tremors occurring in the patient's jaw, chin, or mouth. Additionally, or alternatively, a medical device implanted underneath the patient's skin at the back of the patient's neck may be configured to generate an accelerometer signal that indicates one or more tremors occurring in the patient's torso, limbs, and/or appendages. Tremors may, in some examples, cause movements in one or more areas of the body other than areas where the tremors are centrally located or most prominent. For example, when tremors are most prominent in a patient's hands, this may cause movements that are detectable by a medical device implanted at a back of the patient's neck.

Two or more of a brain activity biomarker, a cardiac activity biomarker, and a motion parameter may be combined to identify one or more symptoms of a patient condition such as Parkinson's disease. For example, an accelerometer signal and an EEG biomarker generated by a sensor device may indicate a level of daytime fatigue, an early-stage symptom of Parkinson's disease. When a patient is fatigued, the patient may have low levels of motion or no motion, and EEG signals may indicate that the patient is in a state of sleep or near sleep. Consequently, a sensor device system may analyze the accelerometer signal and the EEG biomarker to determine a level of daytime fatigue experienced by the patient.

As described in more detail below, the parameter values may be analyzed to detect, predict, or classify one or more conditions based on one or more thresholds, correlation between signals, or using a classification algorithm, which can itself be derived using machine learning techniques applied to databases patient data known to represent the conditions or classifications. The detection algorithm(s) can be passive (involving measurement of a purely resting patient) or active (involving prompting a patient to perform potentially impaired functionality, such as moving particular muscle groups (e.g., raising an arm, moving a finger, moving facial muscles, etc. ,) and/or speaking while recording the electrical response).

Aspects of the technology described herein can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth®)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth® network) on a propagated signal on a propagation medium (e.g., an electromagnetic wave(s), a sound wave) over a period of time, or may be provided on any analog or digital network (packet switched, circuit switched, or other scheme).

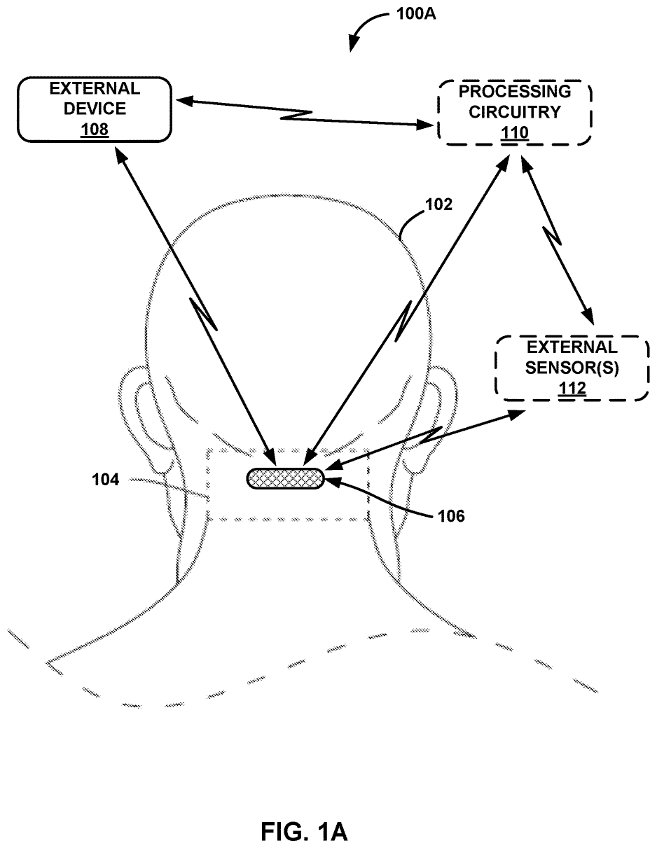

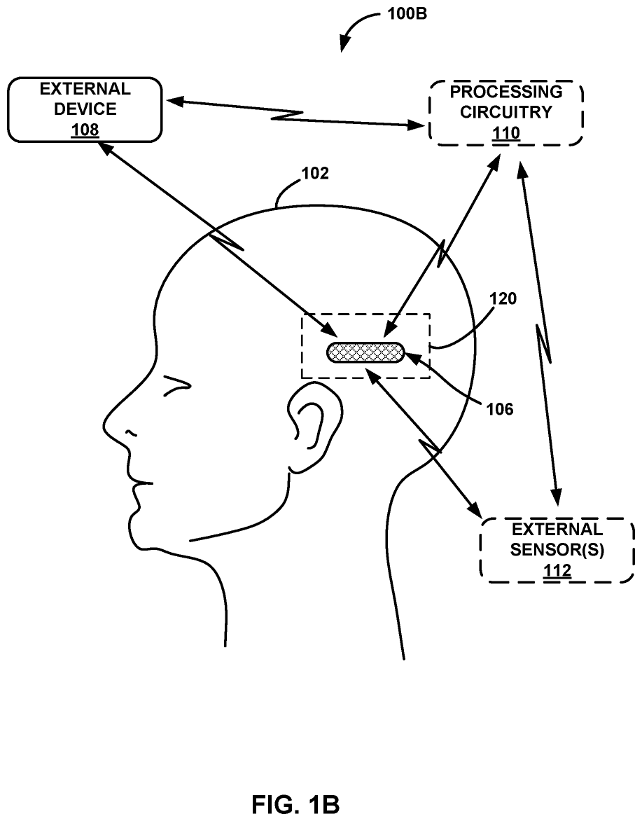

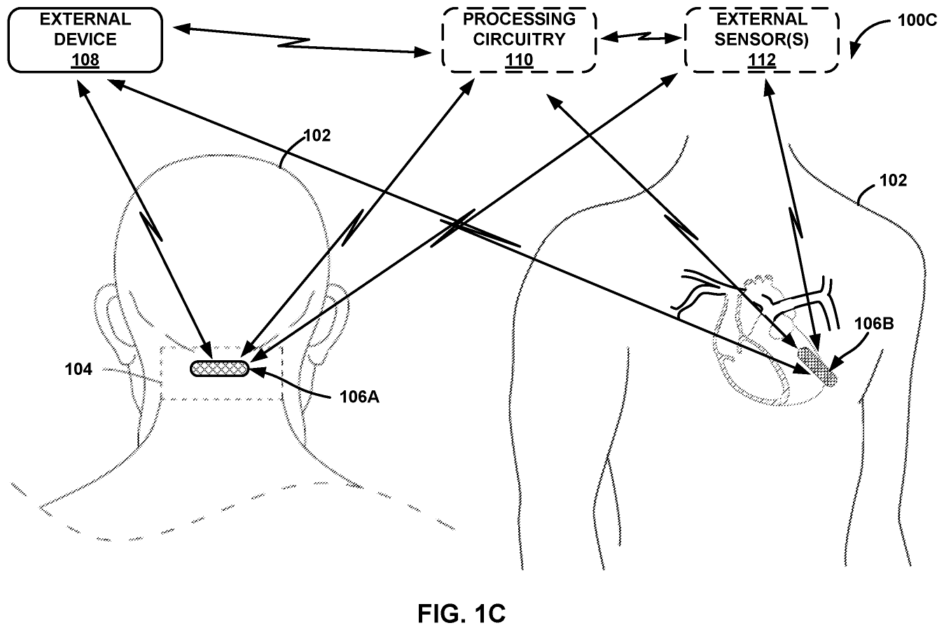

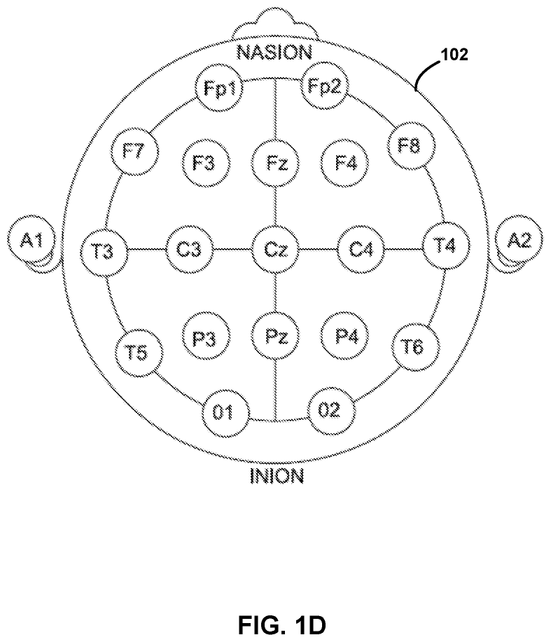

FIG. 1A is a conceptual diagram of a first system 100A configured to detect a medical condition of a patient, in accordance with one or more techniques of this disclosure. The example techniques described herein may be used with a sensor device 106, which in the illustrated example is an implantable medical device (IMD), and which may be in wireless communication with at least one of external device 108, processing circuitry 110, and other devices not pictured in FIG. 1A. For example, an external device (not illustrated in FIG. 1A) may include at least a portion of processing circuitry 110.

As shown in FIG. 1A, sensor device 106 is located in target region 104. Target region 104 can be a rear portion of a user's neck or at the base of the skull. Although sensor device 106 may be implanted at a location generally centered with respect to the head, neck, or target region 104, sensor device 106 may be implanted in an off-center location in order to obtain desired vectors from the electrodes carried on the housing of sensor device 106. Sensor device 106 can be disposed in target region 104 either via implantation (e.g., subcutaneously) or by being placed over the patient's skin with one or more electrodes of sensor device 106 being in direct contact with the patient's skin at or adjacent the target region 104. In some examples, sensor device 106 is configured to be implanted beneath the skin of the patient 102 at a location where a neck of the patient 102 meets a head of the patient 102. In some examples, sensor device 106 is configured to be implanted between a neck muscle of the patient 102 and a skull of the patient 102 In some examples, one or more electrodes of sensor device 106 are configured to face the skull of the patient. In some examples, sensor device 106 is proximate to a brain of patient 102. In some examples, sensor device 106 may be within a range from 1 centimeter (cm) to 3 cm from brain of patient 102.

While conventional EEG electrodes are placed over the patient's scalp and ECG electrodes are positioned elsewhere on the patient's body, the present technology advantageously enables recording of clinically useful brain activity and cardiac activity signals via electrodes positioned at the target region 104 at the rear of the patient's neck or head. This anatomical area is well suited to suited both to implantation of sensor device 106 and to temporary placement of a sensor device over the patient's skin. In contrast, EEG electrodes positioned over the scalp are cumbersome, and implantation over the patient's skull is challenging and may introduce significant patient discomfort.

As noted elsewhere here, conventional EEG electrodes are typically positioned over the scalp to more readily achieve a suitable signal-to-noise ratio for detection of brain activity. However, by using certain digital signal processing, and a special-purpose classifier algorithm, clinically useful brain activity and cardiac activity signals can be obtained using electrodes disposed at the target region 104. Specifically, the electrodes can detect electrical activity that corresponds to brain activity in the P3, Pz, and/or P4 regions (as shown in FIG. 1D).

Processing circuitry 110 may extract values of one or more parameters, e.g., features, from signals indicative of brain activity and/or cardiac activity. Processing circuitry 110 may then determine whether or not the patient has experienced (or has a supra-threshold risk of experiencing) a stroke, epileptic seizure, or other condition based on these parameter value. In some examples, sensor device 106 takes the form of a Reveal LINQ™ or LINQII™ Insertable Cardiac Monitor (ICM), available from Medtronic, Inc., of Minneapolis, Minnesota, or a device has a similar implant volume and similar sensing capabilities. The example techniques may additionally, or alternatively, be used with a medical device not illustrated in FIG. 1A such as another type of IMD, a patch monitor device, a wearable device (e.g., smart watch), or another type of external medical device.

Clinicians sometimes diagnose a patient (e.g., patient 102) with medical conditions and/or determine whether a condition of patient 102 is improving or worsening based on one or more observed physiological signals generated by physiological sensors, such as electrodes, optical sensors, chemical sensors, temperature sensors, acoustic sensors, and motion sensors. In some cases, clinicians apply non-invasive sensors to patients in order to sense one or more physiological signals while a patent is in a clinic for a medical appointment. However, in some examples, events that may change a condition of a patient, such as administration of a therapy, may occur outside of the clinic. As such, in these examples, a clinician may be unable to observe the physiological markers needed to determine whether an event, such as a seizure or stroke, has changed a medical condition of the patient and/or determine whether a medical condition of the patient is improving or worsening while monitoring one or more physiological signals of the patient during a medical appointment. In the example illustrated in FIG. 1A, sensor device 106 is implanted within or attached to patient 102 to continuously record one or more physiological signals of patient 102 over an extended period of time.

In some examples, sensor device 106 includes a plurality of electrodes. Sensor device 106 may sense brain electrical activity and heart electrical activity signals, as well as other signals such as impedance signals for respiration, skin impedance, and perfusion, in some examples. Moreover, sensor device 106 may additionally or alternatively include one or more optical sensors, accelerometers or other motion sensors, temperature sensors, chemical sensors, light sensors, pressure sensors, and acoustic sensors, in some examples. Such sensors may sense various signals that may improve the ability of processing circuitry 110 to detect, predict, or classify patient conditions. In some examples, one or more accelerometers of sensor device 106 may be referred to herein as “accelerometer circuitry.”

External device 108 may be a hand-held computing device with a display viewable by the user and an interface for providing input to external device 108 (e.g., a user input mechanism). For example, external device 108 may include a small display screen (e.g., a liquid crystal display (LCD) or a light emitting diode (LED) display) that presents information to the user. In addition, external device 108 may include a touch screen display, keypad, buttons, a peripheral pointing device, voice activation, or another input mechanism that allows the user to navigate through the user interface of external device 108 and provide input. If external device 108 includes buttons and a keypad, the buttons may be dedicated to performing a certain function, e.g., a power button, the buttons and the keypad may be soft keys that change in function depending upon the section of the user interface currently viewed by the user, or any combination thereof. In some examples, external device 108 is a smartphone of patient 102, which may communicate with sensor device 106, e.g., via Bluetooth™.

In other examples, external device 108 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device. In some examples, external device 108 is configured to communicate with a computer network, such as the Medtronic CareLink® Network developed by Medtronic, plc, of Dublin, Ireland, or another network developed by another developer.

Processing circuitry 110, in some examples, may include one or more processors that are configured to implement functionality and/or process instructions for execution within sensor device 106. For example, processing circuitry 110 may be capable of processing instructions stored in a storage device. Processing circuitry 110 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 110 may include any suitable structure, whether in hardware, software, firmware, or any combination thereof, to perform the functions ascribed herein to processing circuitry 110.

Processing circuitry 110 may represent processing circuitry located within any one or both of sensor device 106 and external device 108. In some examples, processing circuitry 110 may be entirely located within a housing of sensor device 106. In other examples, processing circuitry 110 may be entirely located within a housing of external device 108. In other examples, processing circuitry 110 may be located within any one or combination of sensor device 106, external device 108, and another device or group of devices that are not illustrated in FIG. 1A. As such, techniques and capabilities attributed herein to processing circuitry 110 may be attributed to any combination of sensor device 106, external device 108, and other devices that are not illustrated in FIG. 1A.

Medical device system 100A of FIG. 1A is an example of a system configured to sense signals and generate detection, predictions, or classifications of patient conditions according to one or more techniques of this disclosure. In some examples, the sensed signals may include features representative of heart function such as depolarizations and repolarizations of the heart. Information relating to the aforementioned events, such as time separating one or more of the events, may be applied by processing circuitry 110 for a number of purposes. Processing circuitry 110 may perform signal processing techniques to extract information indicating the one or more parameters of the cardiac signal. In other some examples, the sensed electrical signals may include features representative of brain function, such as amplitudes of frequencies in one or more frequency bands, such as alpha bands, beta bands, or gamma bands. Processing circuitry 110 may perform various processing circuitry to extract these brain features from the sensed electrical signals.

In some examples, sensor device 106 includes one or more accelerometers or other motion sensors. An accelerometer of sensor device 106 may generate an accelerometer signal which reflects a measurement of any one or more of a motion of patient 102, a posture of patient 102 and a body angle of patient 102. In some cases, the accelerometer may generate a three-axis accelerometer signal indicative of patient 102's movements within a three-dimensional Cartesian space. For example, the accelerometer signal may include a vertical axis accelerometer signal vector, a lateral axis accelerometer signal vector, and a frontal axis accelerometer signal vector. The vertical axis accelerometer signal vector may represent an acceleration of patient 102 along a vertical axis, the lateral axis accelerometer signal vector may represent an acceleration of patient 102 along a lateral axis, and the frontal axis accelerometer signal vector may represent an acceleration of patient 102 along a frontal axis. In some cases, the vertical axis substantially extends along a torso of patient 102 when patient 102 from a neck of patient 102 to a waist of patient 102, the lateral axis extends across a chest of patient 102 perpendicular to the vertical axis, and the frontal axis extends outward from and through the chest of patient 102, the frontal axis being perpendicular to the vertical axis and the lateral axis.

Sensor device 106 may measure other signals an impedance (e.g., subcutaneous impedance) which may indicate respiration, skin impedance, or prefusion, heart sound signals, ballistocardiogram signals, pressure signals, or the like. Processing circuitry 110 may analyze any one or more of the set of parameters in order to determine whether or not patient 102 is experiencing or has a supra-threshold risk of experiencing a conditions, such as stroke or seizure.

In some examples, one or more sensors (e.g., electrodes, motion sensors, optical sensors, temperature sensors, pressure sensors, or any combination thereof) of sensor device 106 may generate a signal that indicates a parameter of a patient. In some examples, the signal that indicates the parameter includes a plurality of parameter values, where each parameter value of the plurality of parameter values represents a measurement of the parameter at a respective interval of time. The plurality of parameter values may represent a sequence of parameter values over time, where each parameter value of the sequence of parameter values are generated by sensor device 106 for each time interval of a sequence of time intervals. For example, sensor device 106 may perform a parameter measurement in order to determine a parameter value of the sequence of parameter values according to a recurring time interval (e.g., every day, every night, every other day, every twelve hours, every hour, every second, or any other recurring time interval). In this way, sensor device 106 may be configured to track a respective patient parameter more effectively as compared with a technique in which a patient parameter is tracked during patient visits to a clinic, since sensor device 106 is implanted within patient 102 and is configured to perform parameter measurements according to recurring time intervals without missing a time interval or performing a parameter measurement off schedule.

Sensor device 106 may be referred to as a system or device. In one example, sensor device 106 may include a plurality of electrodes carried by the housing of sensor device 106, sensing circuitry configured to sense, via at least two electrodes of the plurality of electrodes, electrical signals from patient 10, and a motions sensor, e.g., accelerometer, configured to sense a motion signals of the patient. Sensor device 106 may also include processing circuitry 110. The housing of sensor device 106 carries the plurality of electrodes and contains, or houses, the sensing circuitry, the processing circuitry, the motion sensor, and any other sensors. In this manner, sensor device 106 may be referred to as a leadless sensing device because the electrodes are carried directly by the housing instead of by any leads that extend from the housing.

The signals sensed by sensor device 106 can include electrical brain signals, electrical heart signals, other kinds of biometric signals, or any combination thereof. In some examples, the plurality of electrodes are configured to detect brain signals corresponding to activity in at least one of a P3, Pz, or P4 brain region, which is at the back of the head or upper neck region as shown in FIG. 1D. In this manner, the housing of sensor device 106 may be configured to be disposed at or adjacent a rear portion of a neck or skull base of patient 102. The housing of sensor device 106 may be configured to be implanted within patient 102, such as implanted subcutaneously. In other examples, the housing of sensor device 106 may be configured to be disposed on an external surface of skin of patient 102.

In some examples, sensor device 106 may include a single sensing circuitry configured to generate, from the sensed electrical signals, information that includes both the electrical brain activity data (e.g., EEG data) and the electrical heart activity data (e.g., electrocardiogram (ECG) data). In other examples, the processing circuity of sensor device 106 may include separate hardware that generates different information from the sensed electrical signals. For example, sensor device 106 may include first circuitry configured to generate the electrical brain activity from the electrical signals and second circuitry different from the first circuitry and configured to generate the electrical heart activity data from the electrical signals. Even with the first and second circuitry configured to generate different information, or data, in some examples, sensed electrical signals may be conditioned or processed by one or more electrical components (e.g., filters or amplifiers) prior to being processed by the first and second circuitry. In some examples, parameters determined from electrical brain activity signals data may include features, such as spectral features, indicative of the strength of signals in various frequency bands or at various frequencies.

In some examples, sensor device 106 may include one or more accelerometers or other motion sensors within the housing. The accelerometer may be configured to generate motion data representative of motion of patient 102. Processing circuitry 110 may then be configured to generate the detection, prediction, or classification of one or more conditions based on the motion signal, e.g., in condition with the parameter values determined from the brain and cardiac signals. For example, body motion, or lack thereof, may be indicative of a type of seizure experienced by patient 102. As another example, certain body motions or behaviors (e.g., patterns of motion) may be indicative of stroke. In one example, the processing circuitry 110 may be configured to determine, based on the motion data, that patient 102 has fallen, or has nearly fallen. In response to determining that patient 102 has fallen, the processing circuitry 110 may be configured to inform or modify an algorithm for detecting or predicting stroke or another patent condition. In some examples, stroke may cause a patient to fall. Therefore, in combination with other features extracted from sensed electrical signals, processing circuitry 110 may determine from the fall indication that the stroke metric indicates detection of a stroke. In other examples, sensor device 106 or processing circuitry 110 may determine that a characteristic of the motion data exceeds a threshold. The threshold may be an acceleration value indicative of a fall, for example. For seizure, as another example, a frequency of the motion exceeding a frequency threshold may be indicative of body movement from a seizure.

In some examples, the one or more accelerometers of sensor device 106 may be configured to generate an accelerometer signal that indicates one or more tremor motions associated with Parkinson's disease. Tremors associated with Parkinson's disease may occur in the patient's limbs, extremities, torso, neck, head, or any combination thereof. Sensor device 106 may be configured to detect one or more tremors associated with Parkinson's disease, even if the one or more tremors are centered away from the neck of the patient (e.g., centered in the patient's limbs). In some examples, sensor device 106 may be configured to filter an accelerometer signal generated by one or more accelerometers of sensor device 106 to identify one or more tremors associated with Parkinson's disease.

Processing circuitry 110 may extract various features from the cardiac signal sensed by sensor device 106, e.g., an ECG signal, such as heart rate, heart rate variability, etc. Such cardiac parameters may indicate an autonomic activity state of patient 102, and may inform the detection, prediction, and/or classification of a variety of patient conditions. For example, processing circuitry 110 may classify a seizure as one of a plurality of seizure types based on such parameters. For example, seizure types may include single seizure, stroke induced seizure, epileptic seizure, non-epileptic episodes (such as VVS or psychogenic attacks), absence seizures, tonic-clonic or convulsive seizures, atonic seizures, clonic seizures, tonic seizures, and myoclonic seizures. In some examples, processing circuitry 110 may also determine the seizure type based on accelerometer data, temperature data, or any other parameter extracted from one or more sensors.

In some examples, sensor device 106 may sense, via one or more electrodes, a biometric signal that indicates cardiac activity of the patient 102. In some examples, the biometric signal may include an ECG, but this is not required. In some examples, the biometric signal may indicate a level of α-synuclein in a tissue area of the patient 102 proximate to sensor device 106. In some examples, the level of α-synuclein in a tissue area of the patient 102 proximate to sensor device 106 may indicate a status of Parkinson's disease in patient 102. In some examples, the level of α-synuclein may represent a surrogate parameter for a cardiac potential signal such as an ECG or an EGM. For example, the level of α-synuclein in a tissue area proximate to the sensor device 106 may indicate one or more cardiac events (e.g., atrial depolarizations, ventricular depolarizations) that are also indicated by electrical potential signals.

In some examples, sensor device 106 may sense, via one or more electrodes of the plurality of electrodes, a biometric signal that indicates electrical brain activity of patient 102. In some examples, the biometric signal may represent an EEG signal. In some examples, an EEG may include one or more frequency bands including a delta frequency band including frequencies within a range from 0.5 Hz to 4 Hz, a theta frequency band including frequencies within a range from 4 Hz to 8 Hz, an alpha frequency band including frequencies within a range from 8 Hz to 13 Hz, a beta frequency band including frequencies within a range from 13 Hz to 30 Hz, a gamma frequency band including frequencies within a range from 30 Hz to 80 Hz, or other frequency bands.

In some examples, one or more symptoms of a patient disorder may be associated with oscillations of bioelectrical brain activity at a particular frequency or frequency band. For example, one or more symptoms of Parkinson's disease may be indicated by oscillation in the beta frequency band. In some examples, the oscillation of bioelectric brain signals at a particular frequency or frequency band or range may be associated with one or more symptoms of a patient disorder. These oscillations may be referred to as pathological signals or pathological frequencies. For example, bioelectric brain signals oscillating in the particular frequency range may be associated with one or more symptoms of a patient disorder in the sense that such symptoms frequently occur or manifest themselves when the bioelectric brain signals oscillate at such a frequency range. Such occurrences may be a result of the brain signal oscillations within one or more regions of the brain of a patient interfering with the normal function of that region of the brain. As used herein, a frequency or range of frequencies may be referred to as a pathological frequency or pathological frequency range when oscillations of brain signals at such frequency or frequencies are associated in such a manner with one or more symptoms of a patient disorder. Similarly, bioelectric brain signals oscillating at one or more pathological frequencies may be referred to as pathological brain signals.

As one example, in the case of Parkinson's disease, beta frequency oscillations (e.g., between approximately 13 Hertz (Hz) to approximately 30 Hz) in the subthalamic nucleus (STN), globus pallidus interna (GPi), globus pallidus externa (GPe), and/or other areas of the basal ganglia may be associated with one or more motor symptoms including, e.g., rigidity, akenesia, bradykinesia, diskensia, and/or resting tremor. These motor symptoms may be associated with bioelectric brain signals oscillating in the beta frequency range in the sense that such symptoms frequently occur when the bioelectric brain signals oscillate within the beta frequency range. Persistence of oscillation in the beta frequency range may result in oscillatory “interference” that can limit the normal functions of the above regions of the brain. Networks of oscillating neurons may be synchronized by electrical and chemical signals that cause the activity of the network to phase lock and resonate at some frequency. In some examples, the symptoms of Parkinson's disease generally manifest themselves in conjunction with the presence of beta frequency range oscillations (e.g., above some threshold activity level). In some examples, the frequency of symptom manifestations may increase in conjunction with the presence of beta frequency range oscillations.

Processing circuitry 110 may monitor the biometric signal that indicates electrical brain activity of patient 102 (e.g., the EEG signal) in order to monitor one or more patient conditions such as Parkinson's disease. Since one or more frequency bands of the biometric signal (e.g., the beta frequency band) may indicate one or more symptoms of Parkinson's disease, it may be beneficial for processing circuitry 110 to monitor the biometric signal to track Parkinson's disease.

In some examples, a non-sinusoidal shape of the beta frequency band in the sensorimotor cortex may indicate whether patient 102 is on a medication for Parkinson's disease or off a medication for Parkinson's disease. A change in waveform shape of the beta frequency band may, in some examples, reflect hypersynchronous input originating from the basal ganglia. In other words, a waveform shape of the beta frequency band may represent an electrophysiological biomarker of a state of Parkinson's disease of patient 102. Processing circuitry 110 may monitor the beta frequency band in order to assess an efficacy of one or more treatments delivered to patient 102, monitor a state of Parkinson's disease in patient 102, or diagnose one or more patient conditions such as Parkinson's disease.

In some examples, sensor device 106 may sense, via one or more electrodes of the plurality of electrodes, a biometric signal that indicates cardiac activity of the patient. Although sensor device 106 is implanted at the rear portion of the neck or skull base of patient 102, sensor device 106 may sense the biometric signal that indicates cardiac activity of the patient. For example, α-synuclein deposition may be increased in cutaneous sympathetic adrenergic fibers and sympathetic cholinergic fibers of a patient who has Parkinson's disease as compared with a patient who does not have Parkinson's disease. Sensor device 106 may sense, via the one or more electrodes, a biometric signal that indicates a level of α-synuclein deposition in cutaneous sympathetic adrenergic fibers and sympathetic cholinergic fibers of patient 102 in order to track one or more patient conditions (e.g., track Parkinson's disease). In some examples, α-synuclein deposition might not be increased in sensory fibers of a patient who has Parkinson's disease as compared with a patient who does not have Parkinson's disease.

In some examples, the level of α-synuclein in a tissue area proximate to the sensor device 106 may represent a surrogate parameter for a cardiac potential signal such as an ECG or an EGM. A surrogate parameter may represent a measurable parameter (e.g., a level of chemical concentration, an electrical potential level, or another parameter) that is directly or indirectly correlated with another parameter. For example, the level of α-synuclein in the tissue area proximate to the sensor device 106 may indicate one or more cardiac events that are also indicated by a cardiac potential signal such as an ECG or an EGM. By measuring a surrogate parameter, a medical device system may be configured to monitor a patient condition based on the surrogate parameter instead of monitoring the patient condition using one or more parameters correlated with the surrogate parameter.

Higher α-synuclein deposition may be associated with a higher level of autonomic dysfunction and more advanced Parkinson's disease as compared with lower α-synuclein deposition. Parkinson's disease may affect a patient's autonomic cardiac function, blood pressure, breathing, and body temperature. In some examples, autonomic cardiac function impairment may manifest in heart rate variability, and autonomic respiratory function impairment may manifest in R-wave interval variability. Since heart rate variability and R-wave interval variability may be determined based on a cardiac signal of patient 102; a cardiac signal of patient 102 may serve as a biomarker for Parkinson's Disease. By measuring a signal that indicates α-synuclein deposition in cutaneous autonomic nerves of the patient 102, sensor device 106 may sense a biomarker for monitoring Parkinson's disease.

In some examples, sensor device 106 may sense, using accelerometer circuitry, an accelerometer signal that indicates motion activity of patient 102. In some examples, the motion signal may indicate one or more symptoms of Parkinson's disease such as tremors and/or bradykinesia. Processing circuitry 110 may be configured to process the accelerometer signal to track a progression of patient 102's Parkinson's disease over a period of time. A progression of Parkinson's Disease may refer to a change in a status of Parkinson's Disease over a period of time. In some examples, processing circuitry 110 may process the accelerometer signal to determine that a frequency of an occurrence and/or a severity of tremors and/or bradykinesia are increasing over a period of time. This may indicate that a status of the Parkinson's disease of patient 102 is worsening. In some examples, processing circuitry 110 may process the accelerometer signal to determine that a frequency of an occurrence and/or a severity of tremors and/or bradykinesia remain steady over a period of time. This may indicate that a status of the Parkinson's disease of patient 102 remains steady over the period of time. In some examples, processing circuitry 110 may process the accelerometer signal to determine that a frequency of an occurrence and/or a severity of tremors and/or bradykinesia are decreasing over a period of time. This may indicate that a status of the Parkinson's disease of patient 102 is improving over the period of time.

Sensor device 106 may, in some examples, include an ambulatory noise detection sensor (e.g., an accelerometer) to identify one or more conditions that are beneficial for tracking Parkinson's disease of the patient 102. For example, parameters that are measured while patient 102 is at rest may better indicate a status of Parkinson's disease as compared with parameters that are measured while patient 102 is active. Processing circuitry 110 may analyze an accelerometer signal generated by one or more accelerometers in order to determine whether patient 102 is at rest or active. Processing circuitry 110 may, in some cases, determine a status of Parkinson's disease of patient 102 based on one or more parameters that are collected when the patient is at rest. In some examples, processing circuitry 110 may determine a status of Parkinson's disease of patient 102 by weighting parameters collected when the patient 102 is active differently than parameters collected when patient 102 is at rest.

Symptoms associated with Parkinson's disease may, in some examples, be indicated by a combination of two or more of a brain activity signal, a cardiac activity signal, and a motion activity signal. For example, drowsiness, sleepiness, or fatigue of the patient 102 may result in an accelerometer signal generated by sensor device 106 to indicate low levels of activity and an EEG signal sensed by the sensor device 106 to indicate a state of fatigue in the patient 102. Processing circuitry 110 may analyze the accelerometer signal and the EEG signal to identify a level of daytime sleepiness in patient 102. For example, when the accelerometer signal indicates low levels of activity and the EEG signal indicates fatigue during daytime hours, processing circuitry 110 may indicate that the patient 102 is experiencing daytime sleepiness, drowsiness, or fatigue.

In some examples, processing circuitry 110 may determine, based on the biometric signal that indicates electrical brain activity of patient 102, the biometric signal that indicates cardiac activity of the patient 102, and the accelerometer signal that indicates the motion activity of the patient 102, a patient status that indicates a progression of Parkinson's disease over a period of time. In some examples, it may be beneficial for processing circuitry 110 to monitor the biometric signal that indicates electrical brain activity of patient 102, the biometric signal that indicates cardiac activity of the patient 102, and the accelerometer signal that indicates the motion activity of the patient 102 which are all collected by the same sensor device 106 implanted subcutaneously and proximate to the brain of a patient. In some examples, sensor device 106 may be implanted with a procedure that is less invasive than procedures for implanting many other devices. This means that it may be beneficial for patient 102 to be implanted with sensor device 106 so that sensor device 106 may collect signals over a long period of time so that processing circuitry 110 may track the Parkinson's disease of patient 102 over a long period of time.

To determine the patient status that indicates a progression of Parkinson's disease over a period of time, processing circuitry 110 may, in some examples, track the biometric signal that indicates electrical brain activity of patient 102, the biometric signal that indicates cardiac activity of the patient 102, and the accelerometer signal that indicates the motion activity of the patient 102 in order to determine whether a status of the Parkinson's disease of patient 102 is worsening, improving, or remaining substantially the same over the period of time. For example, when the accelerometer signal that indicates that patient tremors are increasing in frequency and/or severity over the period of time, processing circuitry 110 may determine that the Parkinson's disease of patient 102 is worsening.

In some examples, to sense the biometric signal that indicates electrical brain activity of patient 102, the sensor device 106 is configured to sense an EEG signal that indicates the electrical brain activity of the patient 102 in a tissue region of the brain of the patient. In some examples, sensor device 106 may filter the EEG signal to identify one or more frequency bands of the EEG signal. For example, sensor device 106 may include one or more high-pass filters, low-pass filters, band-pass filters, or any combination thereof that are configured to process the EEG signal to identify the one or more frequency bands. Sensor device 106 may be configured to store the one or more frequency bands. In some examples, a waveform of a frequency band of the one or more frequency band may indicate a progression of Parkinson's disease.

In some examples, the frequency band that indicates the progression of Parkinson's disease represents the beta frequency band comprising a range of frequencies from 13 Hz to 30 Hz, but this is not required. In some examples, the frequency band that indicates the progression of Parkinson's disease comprises a frequency band other than the beta frequency band. In some examples, to determine the patient status of patient 102, processing circuitry 110 may be configured to identify a change in a waveform of the frequency band of the EEG signal over a period of time. Processing circuitry 110 may determine that the change in the waveform of the frequency band of the EEG signal corresponds to a change in the patient status that indicates the progression of Parkinson's disease.

Sensor device 106 may sense an EEG signal that indicates electrical brain activity of patient 102 in a tissue region of the brain of patient 102. In some examples, sensor device 106 may filter the EEG signal to identify a set of frequency bands the EEG signal. In some examples, a waveform of a first frequency band of the set of frequency bands is indicative of a patient status that indicates the progression of Parkinson's disease. The first frequency band of the EEG signal that indicates the progression of Parkinson's disease may, in some examples, include the beta frequency band comprising a range of frequencies from 13 Hz to 30 Hz. In some examples, sensor device 106 may filter the EEG signal to identify a second frequency band of the set of frequency bands. The second frequency band may, in some examples, include a gamma frequency band that comprises a range of frequencies from 30 Hz to 80 Hz. Processing circuitry 110 may identify an amplitude and a phase of the first frequency band of the EEG signal and identify an amplitude and a phase of the second frequency band of the EEG signal. Processing circuitry 110 may determine, based on the amplitude and the phase of the first frequency band and the amplitude and the phase of the second frequency band, whether phase-amplitude coupling exists between the first frequency band and the second frequency band. In some examples, a level of phase-amplitude coupling between the beta frequency band and the gamma frequency band of the EEG signal may indicate a status of Parkinson's disease of patient 102.

Sensor device 106 may, in some cases, include one or more embedded artificial intelligence (AI) processing units to that perform enhanced signal processing techniques such as blind source separation (BSS) and adaptive neural network signal processing. In some examples, the one or more AI processing units may be part of processing circuitry 110, but this is not required. In some examples, the one or more AI processing units may be located as part of sensor device 106 and separate from processing circuitry 110. The one or more AI processing units may use BSS to separate one or more signals from a set of mixed signals. For example, one or more AI processing units may use BSS to separate an EEG from a set of mixed signals sensed via one or more electrodes of sensor device 106. Additionally, or alternatively, one or more AI processing units may use BSS to separate signals indicating a level of α-synuclein in a tissue region of the patient 102 from a set of mixed signals. AI processing units may perform one or more signal processing techniques that process signals with little to no information concerning the signals. For example, BSS and/or adaptive neural network signal processing may isolate EEG signals without information concerning characteristics of EEG signals (e.g., frequency, amplitude).

In some examples, sensor device 106 may include an implantable vital sign monitor that provides additional data indicating a status of Parkinson's disease. In some examples, an implantable medical device other than sensor device 106 may include an implantable vital sign monitor that provides additional data indicating a status of Parkinson's disease. In some examples, system 100A may include a wearable device (not illustrated in FIG. 1A) including a transcutaneous sensor configured to sense one or more signals indicating a status of Parkinson's disease. For example, the wearable device may include one or more of external sensor(s) 112 including a continuous glucose monitoring (CGM) sensor, a levodopa (L-DOPA) sensor, an α-synuclein sensor, or any combination thereof.

In some examples, the implantable vital sign monitor of sensor device 106 may be used to titrate L-DOPA for Parkinson's disease control and recommend patients for deep brain stimulation (DBS) therapy. A patient may, in some cases, receive the implantable monitor when diagnosed with Parkinson's disease. An application may inform patient 102 when to take a dose of L-DOPA. The monitor may detect drug dosing using a chemical sensor to ensure drug compliance. The monitor may detect when the drugs are not effective and adjust drug frequency. The monitor may recommend patient 102 for DBS surgery.

Parkinson's disease monitoring and management may, in some examples, provide evidence that a patient's drug therapy regime is not effective and that the patient should seek interventional therapy (e.g., DBS therapy). Additionally, or alternatively, Parkinson's disease monitoring may allow a system to titrate a patient's drug regimen during a pre-DBS phase of Parkinson's disease to improve a quality of life of the patient. Parkinson's disease progression may, in some examples, be quantified, aggregated, and reported as a single trend towards slowing or halting the progression.

In some examples, system 100A includes one or more external sensor(s) 112. External sensors(s) 112 may, in some examples, be part of a wearable device or another kind of external device, but this is not required. In some examples, external sensors(s) 112 may include one or more standalone external sensors. External sensor(s) 112 may be configured to communicate with one or both of sensor device 106 and processing circuitry 110. In some examples, external sensor(s) 112 include a levodopa (L-dopa) sensor configured to sense a biometric signal that indicates a level of L-dopa present in a tissue area of the patient 102. Processing circuitry 110 may determine a patient status that indicates the progression of Parkinson's disease over the period of time in part based on the level of L-dopa present in the tissue area of the patient 102. The techniques of this disclosure are not limited to tracking a progression of Parkinson's Disease over a period of time. In some examples, system 100A may determine a current status of Parkinson's Disease at a point in time (e.g., detecting a tremor) without tracking a progression of Parkinson's Disease over a period of time.

FIG. 1B is a conceptual diagram illustrating a second system 100B configured to detect a medical condition of patient 102, in accordance with one or more techniques of this disclosure. System 100B may be substantially similar to system 100A of FIG. 1A. However, sensor device 106 of system 100B may be configured to be implanted in target region 120 which is located on the side of the head posterior of the temple of patient 102. In some examples, sensor device 106 of system 100B may be implanted submuscularly proximate to one or more temporal lobes of patient 102. Sensor device 106 implanted at target region 120 may be configured to sense cardiac electrical signals, brain electrical signals, motion signals, as well as other sensor signals described herein, in this area. In some examples, sensor device 106 may need to employ different filters or other processing or signal conditioning techniques than those at target region 104 due to different types of noise at target region 120, such as muscle activity due to mandible movement or other types of electrical activity. In other examples, sensor device 106 may be configured to sense signals as described herein from other areas of the head of patient 102 that may be outside of target regions 104 and 120. In some examples, system 100B may include one or more sensor devices in addition to sensor device 106. For example, in addition to sensor device 106 located on a left side of the head of patient 102, system 100B may include another sensor device (not illustrated in FIG. 1B) located on a right side of the head of patient 102. In examples where system 100B includes a sensor device on both sides of the patient's head, both sensor devices may be configured to sense cardiac electrical signals, brain electrical signals, motion signals, as well as other sensor signals described herein.

FIG. 1C is a conceptual diagram illustrating a third system 100C configured to detect a medical condition of patient 102, in accordance with one or more techniques of this disclosure. System 100C may be substantially similar to system 100A of FIG. 1A, except that system 100C includes a first sensor device 106A disposed at or adjacent a rear portion of a neck or skull base of patient 102 and a second sensor device 106B disposed at or adjacent a torso of the patient 102 proximate to a heart of the patient. In some examples, first sensor device 106A may be substantially the same as sensor device 106 of FIG. 1A. In some examples, first sensor device 106A and second sensor device 106B may each include one or more accelerometers or other motion sensors. The accelerometers of first sensor device 106A and second sensor device 106B may be configured to generate motion data representative of motion of patient 102. In some examples, processing circuitry 110 may analyze accelerometer signals collected by first sensor device 106A and second sensor device 106B in order to monitor a progression of Parkinson's Disease or other movement disorders of patient 102. In some examples, analyzing accelerometer signals collected by first sensor device 106A and second sensor device 106B in order to monitor a progression of Parkinson's Disease may yield a more accurate determination of patient status as compared with systems that do not analyze accelerometer signals from two or more different devices.

In some examples, first sensor device 106A and/or second sensor device 106B may include an implantable vital sign monitor that provides additional data indicating a status of Parkinson's disease. In some examples, system 100C may include a wearable device (not illustrated in FIG. 1C) including a transcutaneous sensor configured to sense one or more signals indicating a status of Parkinson's disease. For example, the wearable device may include a CGM sensor, an L-DOPA sensor, an α-synuclein sensor, or any combination thereof.

FIG. 1D is a diagram of a 10-20 map for electroencephalography (EEG) sensor measurements, in accordance with one or more techniques of this disclosure. As shown in FIG. 1D, various locations on the head of patient 102 may be targeted using the electrodes carried by sensor device 106. At the back of the head, such as in target region 104 of FIG. 1A, sensor device 106 may sense electrical signals at least one of P3, Pz or P4. At the side of the head, such as in target region 120 of FIG. 1B, sensor device 106 may sense electrical signals at least one of F7, T3, or T5 and/or at one or more of F8, T4, or T6.

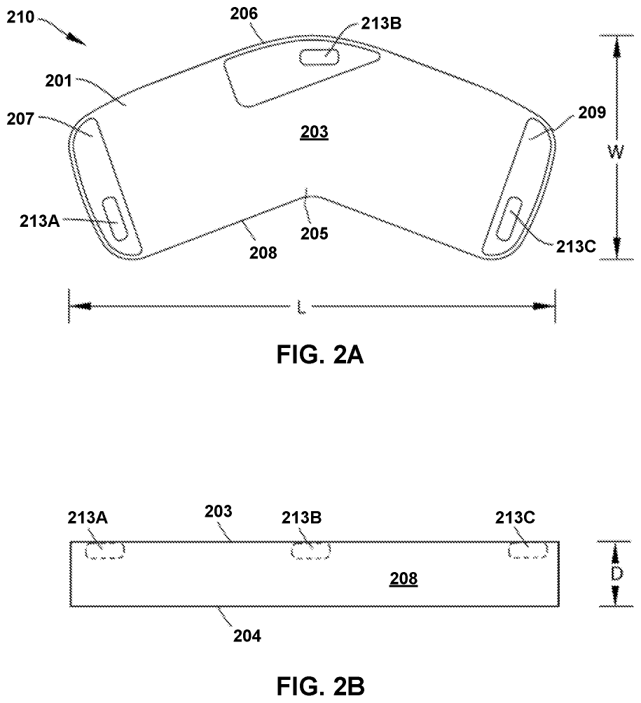

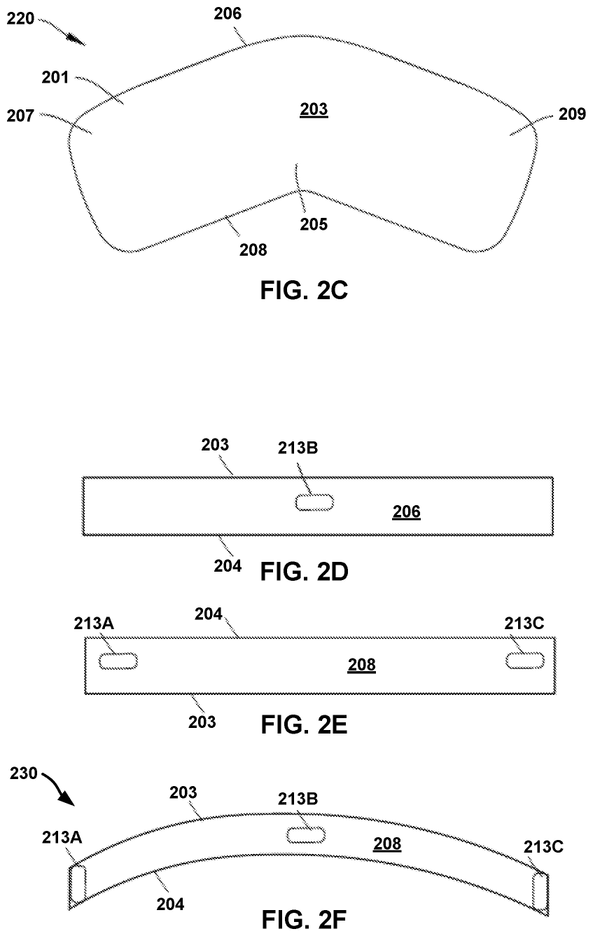

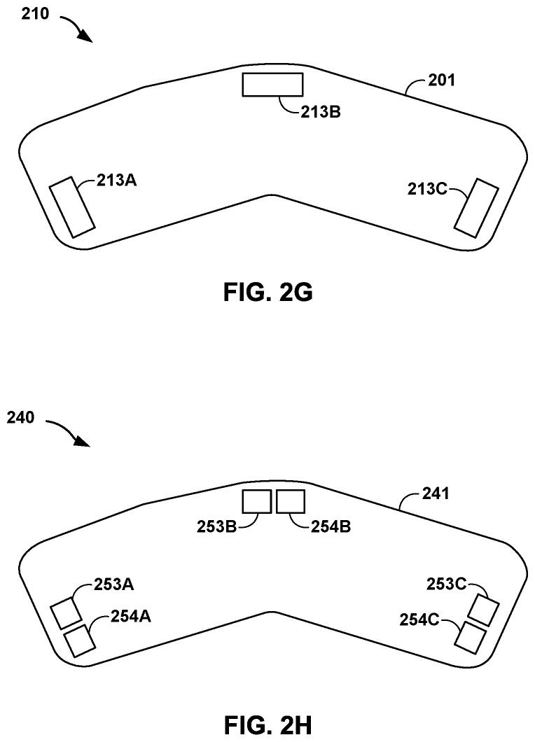

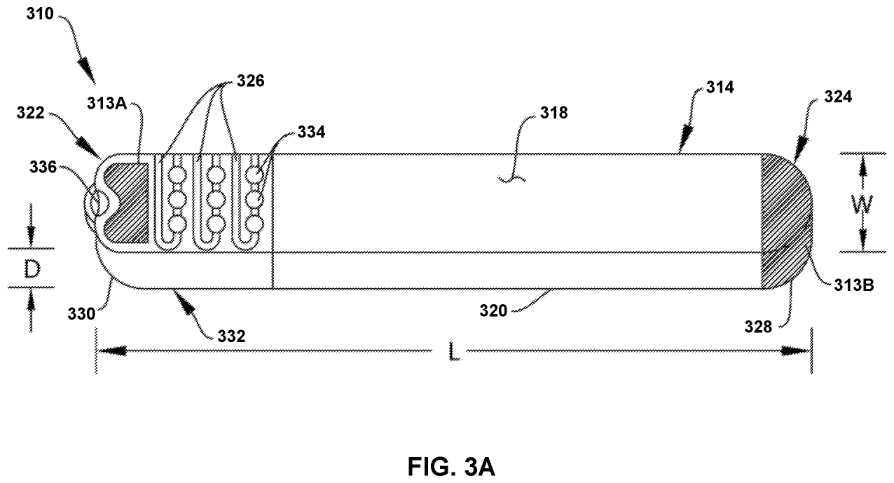

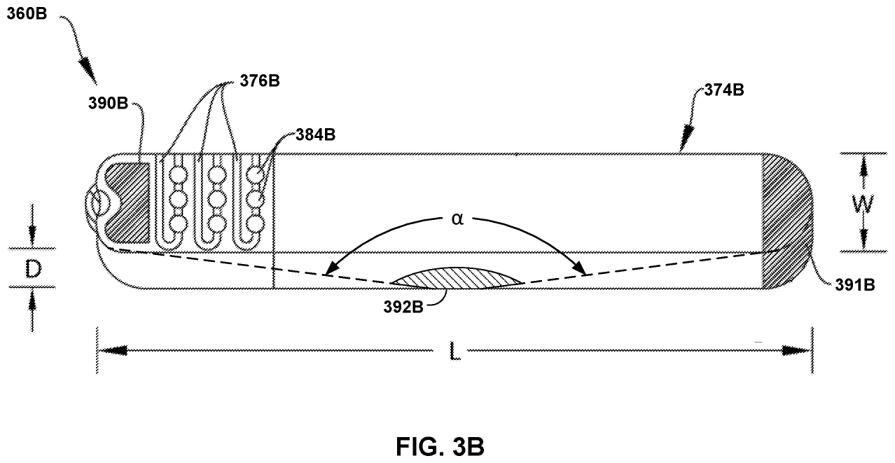

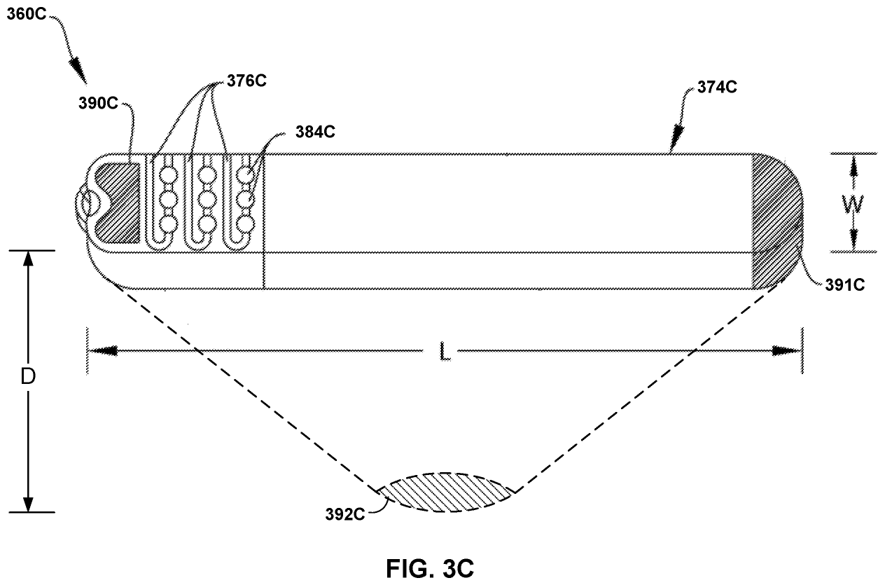

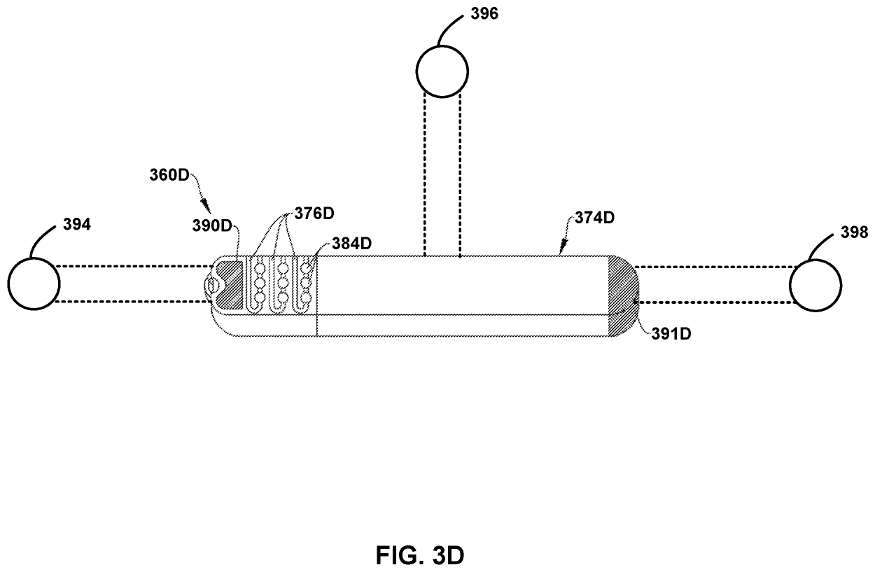

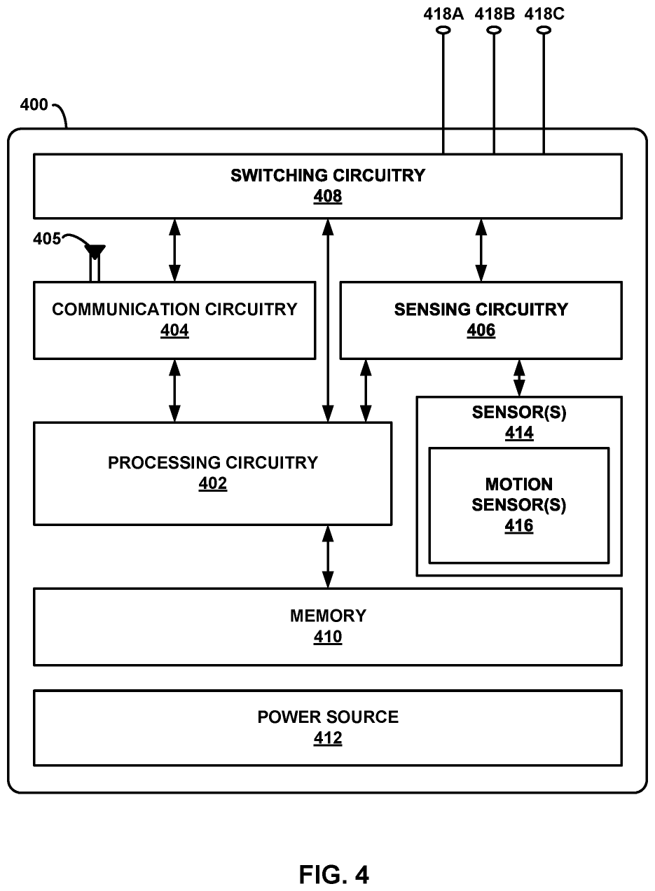

FIG. 2A is a conceptual diagram illustrating a top view of a sensor device 210 (e.g., an IMD), in accordance with one or more techniques of this disclosure. FIG. 2B is a conceptual diagram illustrating a side view of sensor device 210 shown in FIG. 2A, in accordance with one or more techniques of this disclosure. In some examples, sensor device 210 can include some or all of the features of, and be similar to, sensor device 106 described above with respect to FIGS. 1A and 1B and/or the sensor devices 310, 360B, 360B, or 400 described below with respect to FIGS. 3A-3D and 4, and can include additional features as described in connection with FIG. 2A. In the illustrated example, sensor device 210 includes a housing 201 that carries a plurality of electrodes 213A, 213B, and 213C (collectively “electrodes 213”) therein. Although three electrodes are shown for sensor device 210, in other examples, only two electrodes may be carried or four or more electrodes may be carried by housing 201. As shown in FIG. 2H, any of the electrodes may be segmented; that is, each electrode may include two conductive portions separated by an insulative material. In some examples, a first portion may be configured to sense ECG signals, and a second portion may be configured to sense EEG signals.

In operation, electrodes 213 can be placed in direct contact with tissue at the target site (e.g., with the user's skin if placed over the user's skin, or with subcutaneous tissue if the sensor device 210 is implanted). Housing 201 additionally encloses electronic circuitry located inside the sensor device 210 and protects the circuitry (e.g., processing circuitry, sensing circuitry, communication circuitry, sensors, and a power source) contained therein from body fluids. In various examples, electrodes 213 can be disposed along any surface of the sensor device 210 (e.g., anterior surface, posterior surface, left lateral surface, right lateral surface, superior side surface, inferior side surface, or otherwise), and the surface in turn may take any suitable form.

In the example of FIGS. 2A and 2B, housing 201 can be a biocompatible material having a relatively planar shape including a first major surface 203 configured to face towards the tissue of interest (e.g., to face anteriorly when positioned at the back of the patient's neck) a second major surface 204 opposite the first, and a depth D or thickness of housing 201 extending between the first and second major surfaces. Housing 201 can define a superior side surface 206 (e.g., configured to face superiorly when sensing device 210 is implanted in or at the patient's head or neck) and an opposing inferior side surface 208. Housing 201 can further include a central portion 205, a first lateral portion (or left portion) 207, and a second lateral portion (or right portion) 209. Electrodes 213 are distributed about housing 201 such that a central electrode 213B is disposed within the central portion 205 (e.g., substantially centrally along a horizontal axis of the device), a left electrode 213 A electrode is disposed within the left portion 207, and a right electrode 213C is disposed within the right portion 209. As illustrated, housing 201 can define a boomerang or chevron-like shape in which the central portion 205 includes a vertex, with the first and second lateral portions 207 and 209 extending both laterally outward and from the central portion 205 and also at a downward angle with respect to a horizontal axis of the device. In other examples, housing 201 may be formed in other shapes which may be determined by desired distances or angles between different electrodes 213 carried by housing 201.

The configuration of housing 201 can facilitate placement either over the user's skin in a wearable or bandage-like form or for subcutaneous implantation. As such, a relatively thin housing 201 can be advantageous. Additionally, housing 201 can be flexible in some embodiments, so that housing 201 can at least partially bend to correspond to the anatomy of the patient's neck (e.g., with left and right lateral portions 207 and 209 of housing 201 bending anteriorly relative to the central portion 205 of housing 201).

In some embodiments, housing 201 can have a length L of from about 15 to about 50 mm, from about 20 to about 30 mm, or about 25 mm. Housing 201 can have a width W from about 2.5 to about 15 mm, from about 5 to about 10 mm, or about 7.5 mm. In some embodiments, housing 201 can have a thickness of the thickness is less than about 10 mm, about 9 mm, about 8 mm, about 7 mm, about 6 mm, about 5 mm, about 4 mm, or about 3 mm. In some embodiments, the thickness of housing 201 can be from about 2 to about 8 mm, from about 3 to about 5 mm, or about 4 mm. Housing 201 can have a volume of less than about 1.5 cc, about 1.4 cc, about 1.3 cc, about 1.2 cc, about 1.1 cc, about 1.0 cc, about 0.9 cc, about 0.8 cc, about 0.7 cc, about 0.6 cc, about 0.5 cc, or about 0.4 cc. In some embodiments, housing 201 can have dimensions suitable for implantation through a trocar introducer or any other suitable implantation technique.

As illustrated, electrodes 213 carried by housing 201 are arranged so that all three electrodes 213 do not lie on a common axis. In such a configuration, electrodes 213 can achieve a better signal vector as compared to electrodes that are all aligned along a single axis. This can be particularly useful in a sensor device 210 configured to be implanted at the neck or head while detecting electrical activity in the brain and the heart.

In the example shown in FIG. 2B, all three electrodes 213 are located on the first major surface 203 and are substantially flat and outwardly facing. However, in other examples one or more electrodes 213 may utilize a three-dimensional configuration (e.g., curved around an edge of the device 210). Similarly, in other examples one or more electrodes 213 may be disposed on the second major surface opposite the first. The various electrode configurations allow for configurations in which electrodes 213 are located on both the first major surface and the second major surface. In other configurations, such as that shown in FIG. 2B, electrodes 213 are only disposed on one of the major surfaces of housing 201. Electrodes 213 may be formed of a plurality of different types of biocompatible conductive material (e.g., stainless steel, titanium, platinum, iridium, or alloys thereof), and may utilize one or more coatings such as titanium nitride or fractal titanium nitride. In some examples, the material choice for electrodes can also include materials having a high surface area (e.g., to provide better electrode capacitance for better sensitivity) and roughness (e.g., to aid implant stability). Although the example shown in FIGS. 2A and 2B includes three electrodes 213, in some embodiments the sensor device 210 can include 1, 2, 4, 5, 6, or more electrodes carried by housing 201.

FIG. 2C is a conceptual diagram illustrating a top view of another example sensor device 220, in accordance with one or more techniques of this disclosure. FIG. 2C illustrates sensor device 220 which is substantially similar to sensor device 210, but sensor device 220 includes electrodes 213 which are not exposed along the first major surface 203 of housing 201. Instead, electrodes 213 can be exposed along superior and inferior side surfaces (e.g., facing superiorly and inferiorly when implanted at or on a patient's neck), as shown in FIGS. 2D and 2E.

FIG. 2F is a conceptual diagram illustrating a sensor device 230 which is substantially similar to sensor devices 210 and 220, but includes a housing 201 constructed to have a curved configuration, and on which the electrodes can be places along the superior and/or inferior side surfaces of housing 201, in accordance with one or more techniques of this disclosure. In some embodiments, a curved configuration can improve patient comfort and more readily conform to the anatomy of the patient's neck region. In some examples, any of sensor devices 210, 220, or 230 may be flexible in order to conform to the anatomy of the patient at the desired implant or external surface location.

In operation, electrodes 213 are used to sense electrical signals (e.g., EEG or other brain electrical signals and/or ECG or other heart electrical signals) which may be submuscular or subcutaneous. The sensed electrical signals may be stored in a memory of the sensor device, and signal data may be transmitted via a communications link to another device (e.g., external device 108 of FIG. 1A). The sensed electrical signals may be time-coded or otherwise correlated with time data, and stored in this form, so that the recency, frequency, time of day, time span, or date(s) of a particular signal data point or data series (or computed measures or statistics based thereon) may be determined and/or reported. In some examples, electrodes 213 may additionally or alternatively be used for sensing any bio-potential signal of interest, such as electromyogram (EMG) or a nerve signal, as well as impedance signals, from any implanted or external location. These signals may be time-coded or time-correlated, and stored in that form, in the manner described above with respect to brain and cardiac signal data.

FIGS. 2G and 2H depict top views of devices in accordance with examples of the present disclosure. FIG. 2G depicts housing 201 of sensor device 210, which includes electrodes 213A-213C arranged at the perimeter of housing 201. Each of electrodes 213A-213C may be configured to receive raw signals including ECG and EEG components. Sensor device 210 may include circuitry configured to filter the raw signals received by electrodes 213A-213C to generate an ECG signals and EEG signals. In some examples, this circuitry may be located outside of sensor device 210.

FIG. 2H depicts housing 241 of sensor device 240, which includes electrodes 253A-253C and 254A-254C. Electrodes 253A and 254A together may be referred to as a segmented electrode. Similarly, electrodes 253B and 254B may be referred to as a segmented electrode, and electrodes 253C and 254C may be referred to as a segmented electrode. Insulative material may separate the conductive portions (e.g., electrodes 253A and 254A) of a segmented electrode.

Circuitry may be configured to generate a first ECG signal based on a differential signal received at electrodes 253A and 253B, generate a second ECG signal based on a differential signal received at electrodes 253B and 253C, and/or generate a third ECG signal based on a differential signal received at electrodes 253C and 253A. Likewise, the circuitry may be configured to generate a first EEG signal based on a differential signal received at electrodes 254A and 254B, generate a second EEG signal based on a differential signal received at electrodes 254B and 254C, and/or generate a third EEG signal based on a differential signal received at electrodes 254C and 254A.

FIGS. 3A-3D depict other example sensor devices 310, 360B, 360C, and 360D, in accordance with one or more techniques of this disclosure. In some examples, sensor device 310 can include some or all of the features of sensor devices 106, 210, 220, 230, and 400 described herein in accordance with embodiments of the present technology, and can include additional features as described in connection with FIG. 3A. In the example shown in FIG. 3A, sensor device 310 may be embodied as a monitoring device having housing 314, proximal electrode 313A and distal electrode 313B (individually or collectively “electrode 313” or “electrodes 313”). Housing 314 may further comprise first major surface 318, second major surface 320, proximal end 322, and distal end 324. Housing 314 encloses electronic circuitry located inside sensor device 310 and protects the circuitry contained therein from body fluids. Electrical feedthroughs provide electrical connection of electrodes 313. In an example, sensor device 310 may be embodied as an external monitor, such as patch that may be positioned on an external surface of the patient, or another type of medical device (e.g., instead of as an ICM), such as described further herein.

In the example shown in FIG. 3A, sensor device 310 is defined by a length “L,” a width “W,” and thickness or depth “D.” sensor device 310 may be in the form of an elongated rectangular prism wherein the length L is significantly larger than the width W, which in turn is larger than the depth D. In one example, the geometry of sensor device 310—in particular, a width W being greater than the depth D—is selected to allow sensor device 310 to be inserted under the skin of the patient using a minimally invasive procedure and to remain in the desired orientation during insertion. For example, the device shown in FIG. 3 includes radial asymmetries (notably, the rectangular shape) along the longitudinal axis that maintains the device in the proper orientation following insertion. For example, in one example the spacing between proximal electrode 313A and distal electrode 313B may range from 30 millimeters (mm) to 55 mm, 35 mm to 55 mm, and from 40 mm to 55 mm and may be any range or individual spacing from 25 mm to 60 mm. In-some examples, the length L may be from 30 mm to about 70 mm. In other examples, the length L may range from 40 mm to 60 mm, 45 mm to 60 mm and may be any length or range of lengths between about 30 mm and about 70 mm. In addition, the width W of first major surface 18 may range from 3 mm to 10 mm and may be any single or range of widths between 3 mm and 10 mm. The thickness of depth D of sensor device 310 may range from 2 mm to 9 mm. In other examples, the depth D of sensor device 310 may range from 2 mm to 5 mm and may be any single or range of depths from 2 mm to 9 mm. In addition, sensor device 310 according to an example of the present disclosure is has a geometry and size designed for ease of implant and patient comfort. Examples of sensor device 310 described in this disclosure may have a volume of 3 cc or less, 2 cc or less, 1 cc or less, 0.9 cc or less, 0.8 cc or less, 0.7 cc or less, 0.6 cc or less, 0.5 cc or less, or 0.4 cc or less, any volume between 3 and 0.4 cc. In addition, in the example shown in FIG. 3A, proximal end 322 and distal end 324 are rounded to reduce discomfort and irritation to surrounding tissue once inserted under the skin of the patient.

In the example shown in FIG. 3A, once inserted within the patient, the first major surface 318 faces outward, toward the skin of the patient while the second major surface 320 is located opposite the first major surface 318. Consequently, the first and second major surfaces may face in directions along a sagittal axis of patient, and this orientation may be consistently achieved upon implantation due to the dimensions of sensor device 310. Additionally, an accelerometer, or axis of an accelerometer, may be oriented along the sagittal axis.

Proximal electrode 313A and distal electrode 313B are used to sense electrical signals (e.g., EEG signals or ECG signals) which may be submuscular or subcutaneous. Electrical signals may be stored in a memory of sensor device 310, and signal data may be transmitted via integrated antenna 326 to another medical device, which may be another implantable device or an external device, such as external device 108 (FIG. 1A). In some examples, electrodes 313A and 313B may additionally or alternatively be used for sensing any bio-potential signal of interest, such as an electrocardiogram (ECG), intracardiac electrogram (EGM), electromyogram (EMG), or a nerve signal, from any implanted location.

In the example shown in FIG. 3A, proximal electrode 313A is in close proximity to the proximal end 322, and distal electrode 313B is in close proximity to distal end 324. In this example, distal electrode 313B is not limited to a flattened, outward facing surface, but may extend from first major surface 318 around rounded edges 328 or end surface 330 and onto the second major surface 320 so that the electrode 313B has a three-dimensional curved configuration. In the example shown in FIG. 3, proximal electrode 313A is located on first major surface 318 and is substantially flat, outward facing. However, in other examples proximal electrode 313A may utilize the three-dimensional curved configuration of distal electrode 313B, providing a three-dimensional proximal electrode (not shown in this example). Similarly, in other examples distal electrode 313B may utilize a substantially flat, outward facing electrode located on first major surface 318 similar to that shown with respect to proximal electrode 313A. The various electrode configurations allow for configurations in which proximal electrode 313A and distal electrode 313B are located on both first major surface 318 and second major surface 320. In other configurations, such as that shown in FIG. 3, only one of proximal electrode 313A and distal electrode 313B is located on both major surfaces 318 and 320, and in still other configurations both proximal electrode 313A and distal electrode 313B are located on one of the first major surface 318 or the second major surface 320 (e.g., proximal electrode 313A located on first major surface 318 while distal electrode 313B is located on second major surface 320). In another example, sensor device 310 may include electrodes 313 on both first major surface 318 and second major surface 320 at or near the proximal and distal ends of the device, such that a total of four electrodes 313 are included on sensor device 310. Electrodes 313 may be formed of a plurality of different types of biocompatible conductive material (e.g., stainless steel, titanium, platinum, iridium, or alloys thereof), and may utilize one or more coatings such as titanium nitride or fractal titanium nitride. Although the example shown in FIG. 3A includes two electrodes 313, in some embodiments sensor device 310 can include 3, 4, 5, or more electrodes carried by the housing 314.

In the example shown in FIG. 3A, proximal end 322 includes a header assembly 332 that includes one or more of proximal electrode 313A, integrated antenna 326, anti-migration projections 334, or suture hole 336. Integrated antenna 326 is located on the same major surface (i.e., first major surface 318) as proximal electrode 313a and is also included as part of header assembly 332. Integrated antenna 326 allows sensor device 310 to transmit or receive data. In other examples, integrated antenna 326 may be formed on the opposite major surface as proximal electrode 313A, or may be incorporated within the housing 314 of sensor device 310. In the example shown in FIG. 3A, anti-migration projections 334 are located adjacent to integrated antenna 326 and protrude away from first major surface 318 to prevent longitudinal movement of the device. In the example shown in FIG. 3 anti-migration projections 334 includes a plurality (e.g., six or nine) small bumps or protrusions extending away from first major surface 318. As discussed above, in other examples anti-migration projections 334 may be located on the opposite major surface as proximal electrode 313A or integrated antenna 326. In addition, in the example shown in FIG. 3A header assembly 332 includes suture hole 336, which provides another means of securing sensor device 310 to the patient to prevent movement following insert. In the example shown, suture hole 336 is located adjacent to proximal electrode 313A. In one example, header assembly 332 is a molded header assembly made from a polymeric or plastic material, which may be integrated or separable from the main portion of sensor device 310.

FIG. 3B shows a sensor device 360B including a third electrode 392B at a midpoint between electrodes 390B and 391B. As seen in FIG. 3B, sensor device 360B also includes a housing 374B, an integrated antenna 376B, and anti-migration projections 384B. The dimension D of housing 374B of sensor device 360B can be increased to adjust the angle a to obtain a more orthogonal orientation for the triangular configuration of electrodes 390B-392B. In some examples, sensor device 360B may have the same shape and dimensions as sensor device 310, except that electrode 392B is added to the side surface or back surface of housing 374B to create a triangle-shaped electrode configuration.

FIG. 3C shows sensor device 360C with an extended third dimension D. As seen in FIG. 3C, sensor device 360C also includes a housing 374C, an integrated antenna 376C, electrode 390C, electrode 391B, and electrode 392C, a housing 374C, an integrated antenna 376C, and anti-migration projections 384C. Third electrode 392C is positioned at a corner to create a triangular-shaped electrode configuration with electrodes 390C and 391C. Dimension D can be designed to achieve specific angles for the triangular configuration of electrodes 390C-392C.

FIG. 3D shows sensor device 360D having extended electrodes. As seen in FIG. 3D, sensor device 360D also includes a housing 374D, an integrated antenna 376D, electrode 390D, electrode 391D, and electrode 392D, a housing 374D, an integrated antenna 376D, and anti-migration projections 384D. Sensor device 360D also may include any one or combination of electrode 394, electrode 396, and electrode 398. Electrodes 394, 396, 398 may extend outward from housing 374C.

FIG. 4 is a block diagram of an example configuration of a sensor device 400 configured to sense signals used to generate at least one of a detection, prediction, or classification of a condition of a patient, in accordance with one or more techniques of this disclosure. Sensor device 400 may be an example of any of sensor devices 106, 210, 220, 230, 310, 360A, 360B, and 360C. In the illustrated example, sensor device 400 includes processing circuitry 402, communication circuitry 404, antenna 405, sensing circuitry 406, switching circuitry 408, memory 410, power source 412, sensor(s) 414 including motion sensor(s) 416, and electrodes 418A-418C (collectively, “electrodes 418”).

Processing circuitry 402 may include fixed function circuitry and/or programmable processing circuitry. Processing circuitry 402 may include any one or more of a microprocessor, a controller, a DSP, an ASIC, an FPGA, a graphics processing unit (GPU), a tensor processing unit (TPU), or equivalent discrete or analog logic circuitry. In some examples, processing circuitry 402 may include multiple components, such as any combination of one or more microprocessors, one or more controllers, one or more DSPs, one or more ASICs, one or more FPGAs, one or more GPUs, one or more TPUs, as well as other discrete or integrated logic circuitry. The functions attributed to processing circuitry 402 herein may be embodied as software, firmware, hardware or any combination thereof. Processing circuitry 402 may be an example of or component of processing circuitry 110 (FIGS. 1A and 1B). In some examples, processing circuitry 110 is located entirely within processing circuitry 402 of sensor device 400. In some examples, processing circuitry 110 includes processing circuitry 402 and processing circuitry of one or more other devices (e.g., external device 108).

Communication circuitry 404 and sensing circuitry 406 may be selectively coupled to electrodes 418A-418C via switching circuitry 408, as controlled by processing circuitry 402. Communication circuitry 404 may include any suitable hardware, firmware, software or any combination thereof for communicating with another device, such as external device 108. For example, communication circuitry 404 may include appropriate modulation, demodulation, frequency conversion, filtering, and amplifier components for transmission and reception of data. Under the control of processing circuitry 402, communication circuitry 404 may receive downlink telemetry from, as well as send uplink telemetry to, external device 108 or another device with the aid of an internal or external antenna, e.g., antenna 405. Communication circuitry 404 may include any combination of a Bluetooth® radio, an electronic oscillator, frequency modulation circuitry, frequency demodulation circuitry, amplifier circuitry, and power switches such as a metal-oxide-semiconductor field-effect transistors (MOSFET), a bipolar junction transistor (BJT), an insulated-gate bipolar transistor (IGBT), a junction field effect transistor (JFET), or another element that uses voltage for its control.

In some examples, processing circuitry 402 may use communication circuitry 404 to communicate with a networked computing device via an external device (e.g., external device 108) and a computer network, such as the Medtronic CareLink® Network developed by Medtronic, plc, of Dublin, Ireland. A clinician or other user may, in some cases, retrieve data from sensor device 400 using external device 108, or by using another local or networked computing device configured to communicate with processing circuitry 402 via communication circuitry 404. The clinician may also program parameters of sensor device 400 using external device 108 or another local or networked computing device.

Sensing circuitry 406 may monitor signals from electrodes 418A-418C in order to monitor electrical activity of the brain and heart (e.g., to produce an EEG and ECG) from which processing circuitry 402 (or processing circuitry of another device) may determine values over time of parameters used to generate the detection, prediction, or classification. Sensing circuitry 406 may also sense physiological characteristics such as subcutaneous tissue impedance, the impedance being indicative of at least some aspects of patient 102's respiratory patterns or perfusion. In some examples, a subcutaneous impedance signal sensed by sensor device 400 may indicate a respiratory rate and/or a respiratory intensity of patient 102. Sensing circuitry 406 also may monitor signals from sensors 414, which may include motion sensor(s) 416, and any additional sensors, such as light detectors, pressure sensors, or acoustic sensors, that may be positioned on or in sensor device 400. In some examples, sensing circuitry 406 may include one or more filters and amplifiers for filtering and amplifying signals received from one or more of electrodes 418A-418C and/or sensor(s) 414.

Memory 410 may be configured to store information within sensing device 400 during operation. In some examples, memory 410 may be referred to as a storage device and include computer-readable instructions that, when executed by processing circuitry 402, cause sensor device 400 and processing circuitry 402 to perform various functions attributed to sensor device 400 and processing circuitry 402 herein. Memory 410 may include any volatile, non-volatile, magnetic, optical, or electrical media, such as a random access memory (RAM), dynamic random-access memories (DRAM), static random-access memories (SRAM), magnetic discs, optical discs, flash memories, read-only memory (ROM), non-volatile RAM (NVRAM), electrically programmable memories (EPROM), electrically-erasable programmable ROM (EEPROM), flash memory, or any other digital media. Memory 410 may also store data generated by sensing circuitry 406, such as signals, or data generated by processing circuitry 402, such as parameter values or indications of detections, predictions, or classifications of conditions.

Power source 412 is configured to deliver operating power to the components of sensor device 400. Power source 412 may include a battery and a power generation circuit to produce the operating power. In some examples, the battery is rechargeable to allow extended operation. In some examples, recharging is accomplished through proximal inductive interaction between an external charger and an inductive charging coil within external device 108. Power source 412 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

As described herein, sensor device 400 may be configured to sense signals, e.g., via electrodes 418 and sensors 414, for detecting, predicting, and/or classifying one or more patient conditions, such as Parkinson's disease. In some examples, processing circuitry 402 may be configured to calculate parameter values relating to one or more electrical signals received from the electrodes 418, and/or signals from sensors 414. In some examples, processing circuitry 402 may be configured to algorithmically determine the presence or absence of a patient condition, whether the patient has a supra-threshold risk of a condition, or whether a condition is most likely a certain type or has a certain cause, based on the parameter values. In some examples, processing circuitry 402 may employ patient movement information as a part of the detection, prediction, and/or classification of conditions. For example, motion sensor 416 may include one or more accelerometers configured to detect patient movement.

In some examples, wherein sensor device 400 is configured to be implanted subcutaneously and proximate to a brain of a patient (e.g., patient 102). Sensor device 400 may be configured to sense, via electrodes 418, a first biometric signal that indicates electrical brain activity of the patient. In some examples, the first biometric signal may include an EEG signal. In some examples, sensor device 400 may sense, via the one or more electrodes, a second biometric signal that indicates cardiac activity of the patient. In some examples, the second biometric signal may include a level of α-synuclein in a tissue area of the patient proximate to the sensing device. In some examples, it may be beneficial to sense the first biometric signal indicating the brain activity of the patient and the second biometric signal indicating the cardiac activity of the patient using the sensor device 400 that is implanted subcutaneously and proximate to the brain of a patient, because sensor device 400 may be configured to continuously sense both the first biometric signal and the second biometric signal over a period of time. This may allow sensor device 400 to sense data which indicates a progression of a patient condition (e.g., Parkinson's disease) over the period of time.

In some examples, to sense the first biometric signal, sensor device 400 may sense an EEG signal that indicates the electrical brain activity of the patient in a tissue region of the brain of the patient. In some examples, sensor device 400 may filter the EEG signal to identify one or more frequency bands of the EEG signal. In some examples, processing circuitry 402 and/or sensing circuitry 406 may filter the EEG signal to identify the one or more frequency bands. The EEG signal may, in some cases, include a set of frequency bands. Each frequency band of the set of frequency bands may indicate information corresponding to brain activity of the patient. In some examples, a frequency band of the set of frequency bands may indicate information that is not indicated by one or more other frequency bands of the set of frequency bands. This means that it may be beneficial to filter the EEG signal to identify each frequency band of the one or more frequency bands so that processing circuitry 110 may analyze each frequency band of the one or more frequency bands to identify the information indicated by each frequency band of the one or more frequency bands. For example, certain frequency bands of the EEG signal may indicate information corresponding to a specific patient condition such as Parkinson's disease.

In some examples, a waveform of a frequency band of the EEG signal may indicate the progression of Parkinson's disease. In some examples, the frequency band including a waveform that indicates the progression of Parkinson's disease comprises a beta frequency band within a range of frequencies from 13 Hz to 30 Hz. That is, processing circuitry 110 may analyze the beta frequency band over a period of time in order to determine a progression of Parkinson's disease over the period of time. When the waveform of the beta frequency band changes over the period of time, this may indicate that the Parkinson's disease of the patient is progressing. Since sensing device 400 is configured to be implanted subcutaneously near the brain of the patient, sensing device 400 may continuously sense an EEG signal and identify the beta frequency band over a long period of time (e.g., days, weeks, months). This may allow processing circuitry to track the waveform of the frequency band of the EEG signal over a long period of time to monitor a status of the patient's Parkinson's disease over a long period of time. Processing circuitry 110 may identify a change in a waveform of the frequency band of the EEG signal over a period of time. The processing circuitry may determine that the change in the waveform of the frequency band of the EEG signal corresponds to a change in a patient status that indicates the progression of Parkinson's disease.

Processing circuitry 110 may use one or more signal processing techniques to identify parameters corresponding to the waveform of a frequency band of the EEG signal. For example, processing circuitry 110 may identify one or more peaks and one or more troughs in the signal. Processing circuitry 110 may identify a sharpness of one or more peaks and/or troughs, identify a slope of the signal between peaks and troughs, identify an amplitude of the signal, identify a variance in amplitude of the signal, or any combination thereof. Processing circuitry 110 may track one or more parameters of the waveform over a period of time in order to determine whether the waveform is changing.

In some examples a relationship between two or more frequency bands of the set of frequency bands of the EEG signal sensed by sensor device 400 may indicate a progression of a patient condition such as Parkinson's disease. For example, sensor device 400 may filter the EEG signal to identify a first frequency band and a second frequency band. In some examples, the first frequency band comprises the beta frequency band within a range of frequencies from 13 Hz to 30 Hz. In some examples, the second frequency band represents a gamma frequency band that comprises a range of frequencies from 30 Hz to 80 Hz. In some examples, a relationship between the beta frequency band and the gamma frequency band indicates information corresponding to Parkinson's disease of a patient. For example, when phase amplitude coupling exists between the beta frequency band and the gamma frequency band, this may indicate a status of the Parkinson's disease of the patient.

To determine whether phase amplitude coupling exists between two or more signals (e.g., between the beta frequency band and the gamma frequency band of the EEG signal), processing circuitry 110 may identify an amplitude and a phase of the beta frequency band of the EEG signal and identify an amplitude and a phase of the gamma frequency band of the EEG signal. Processing circuitry 110 may determine, based on the amplitude and the phase of the beta frequency band and the amplitude and the phase of the gamma frequency band, whether phase amplitude coupling exists between the beta frequency band and the gamma frequency band. In some examples, phase amplitude coupling between the beta frequency band and the gamma frequency band is indicative of the patient status (e.g., a progression of Parkinson's disease).

Sensor device 400 may sense, via electrodes 418 a biometric signal indicating cardiac activity of the patient. Although sensor device 400 may sense an ECG, an EGM, and/or other cardiac potential signals, sensor device 400 may sense one or more other signals corresponding to cardiac activity of the patient. In some examples, the biometric signal indicating the cardiac activity of the patient comprises a level of α-synuclein in a tissue area of the patient proximate to sensor device 400. Sensor device 400 may, in some examples, filter, based on the level of α-synuclein in the tissue area, the second biometric signal to identify a biomarker that indicates the cardiac activity of the patient. Processing circuitry 110 may identify, based on the biomarker signal that indicates the cardiac activity of the patient, one or more cardiac metrics. Processing circuitry 110 may determine the patient status based on the one or more cardiac metrics identified based on the biomarker signal. In some examples, the one or more cardiac metrics include heart rate, heart rate variability, and beat-to-beat interval variability.

Sensor device 400 may generate, using motion sensor(s) 416, an accelerometer signal that indicates motion activity of the patient. Processing circuitry 110 may determine, based on the accelerometer signal, an amount of tremor motion activity of the patient. Processing circuitry 110 may determine the patient status based on the amount of tremor motion activity of the patient.

In some examples, processing circuitry 110 is configured to determine, based on a first biometric signal indicating the electrical brain activity of the patient, a second biometric signal indicating the cardiac activity of the patient, and the accelerometer signal indicating the motion activity of the patient, a patient status. The patient status may indicate a progression of Parkinson's disease over a period of time. Since biometric brain signals, biometric cardiac signals, and motion signals may each indicate one or more aspects of Parkinson's disease, it may be beneficial for processing circuitry 110 to monitor all three of the metric brain signals, biometric cardiac signals, and motion signals.

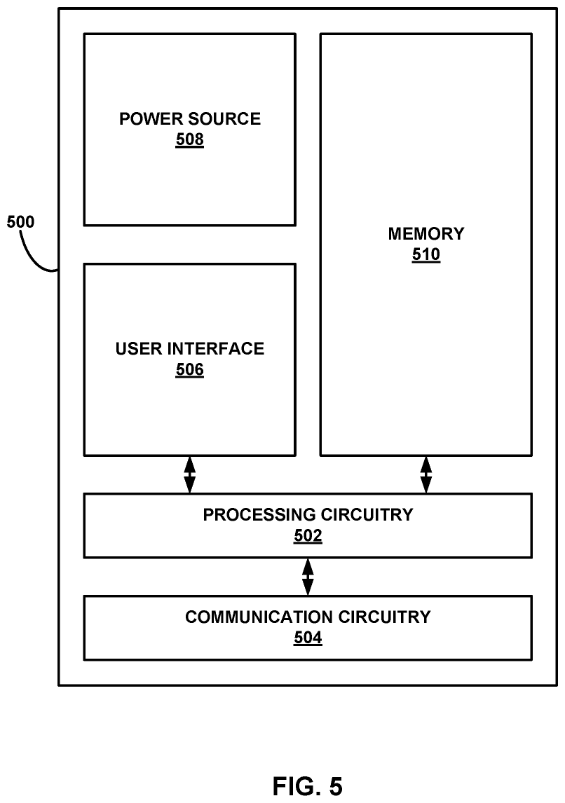

FIG. 5 is a block diagram of an example configuration of an external device 500 configured to communicate with any sensor device (e.g., sensor device 106 or sensor device 400) described herein, in accordance with one or more techniques of this disclosure. External device 500 is an example of external device 108 of FIGS. 1A and 1B. In the example of FIG. 5, external device 500 includes processing circuitry 502, communication circuitry 504, user interface 506, power source 508, and memory 510.

Processing circuitry 502, in one example, may include one or more processors that are configured to implement functionality and/or process instructions for execution within external device 500. For example, processing circuitry 502 may be capable of processing instructions stored in memory 510. Processing circuitry 502 may include, for example, microprocessors, DSPs, ASICs, FPGAs, or equivalent discrete or integrated logic circuitry, or a combination of any of the foregoing devices or circuitry. Accordingly, processing circuitry 502 may include any suitable structure, whether in hardware, software, firmware, or any combination thereof, to perform the functions ascribed herein to processing circuitry 502. Processing circuitry 502 may be an example of or component of processing circuitry 110 (FIGS. 1A and 1B). In some examples, processing circuitry 110 is located entirely within processing circuitry 502 of external device 500. In some examples, processing circuitry 110 includes processing circuitry 502 and processing circuitry of one or more other devices (e.g., sensor device 106).

Communication circuitry 504 may include any suitable hardware, firmware, software or any combination thereof for communicating with another device, such as IMD 400. Under the control of processing circuitry 502, communication circuitry 504 may receive downlink telemetry from, as well as send uplink telemetry to, sensor device 400, or another device.

A user, such as a clinician or patient 102, may interact with external device 500 through user interface 506. User interface 506 includes a display (not shown), such as an LCD or LED display or other type of screen, with which processing circuitry 502 may present information related to sensor device 400 (e.g., biometric data corresponding to a status of Parkinson's disease). In addition, user interface 506 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 502 of external device 500 and provide input. In other examples, user interface 506 also includes audio circuitry for providing audible notifications, instructions or other sounds to patient 102, receiving voice commands from patient 102, or both. Memory 510 may include instructions for operating user interface 506 and for managing power source 508.

Power source 508 is configured to deliver operating power to the components of external device 500. Power source 508 may include a battery and a power generation circuit to produce the operating power. In some examples, the battery is rechargeable to allow extended operation. Recharging may be accomplished by electrically coupling power source 508 to a cradle or plug that is connected to an alternating current (AC) outlet. In addition, recharging may be accomplished through proximal inductive interaction between an external charger and an inductive charging coil within external device 500. In other examples, traditional batteries (e.g., nickel cadmium or lithium ion batteries) may be used. In addition, external device 500 may be directly coupled to an alternating current outlet to operate.

Memory 510 may be configured to store information within external device 500 during operation. In some examples, memory 510 may be referred to as a storage device and include computer-readable instructions that, when executed by processing circuitry 502, cause external device 500 and processing circuitry 502 to perform various functions attributed to external device 500 and processing circuitry 502 herein. Memory 510 may include any volatile, non-volatile, magnetic, optical, or electrical media, such as RAM, DRAM, SRAM, magnetic discs, optical discs, flash memories, ROM, NVRAM, EPROM, EEPROM, flash memory, or any other digital media. Memory 510 may also store data generated by sensing circuitry 406 of sensor device 400, such as signals, parameter values, or indications of detections, predictions, or classifications of conditions.

Data exchanged between external device 500 and sensor device 400 may include operational parameters. External device 500 may transmit data including computer readable instructions which, when implemented by sensor device 400, may control sensor device 400 to change one or more operational parameters and/or export data. For example, processing circuitry 502 may transmit an instruction to sensor device 400 which requests sensor device 400 to export data (e.g., data corresponding to one or more of the sensed signals, parameter values determined based on the signals, or indications that a condition has been detected, predicted, or classified) to external device 500. In turn, external device 500 may receive the data from sensor device 400 and store the data in memory 510. In some examples, external device 500 may provide an alert to the patient or another entity (e.g., a call center) based on a condition detection, prediction, or classification provided by sensor device 400.

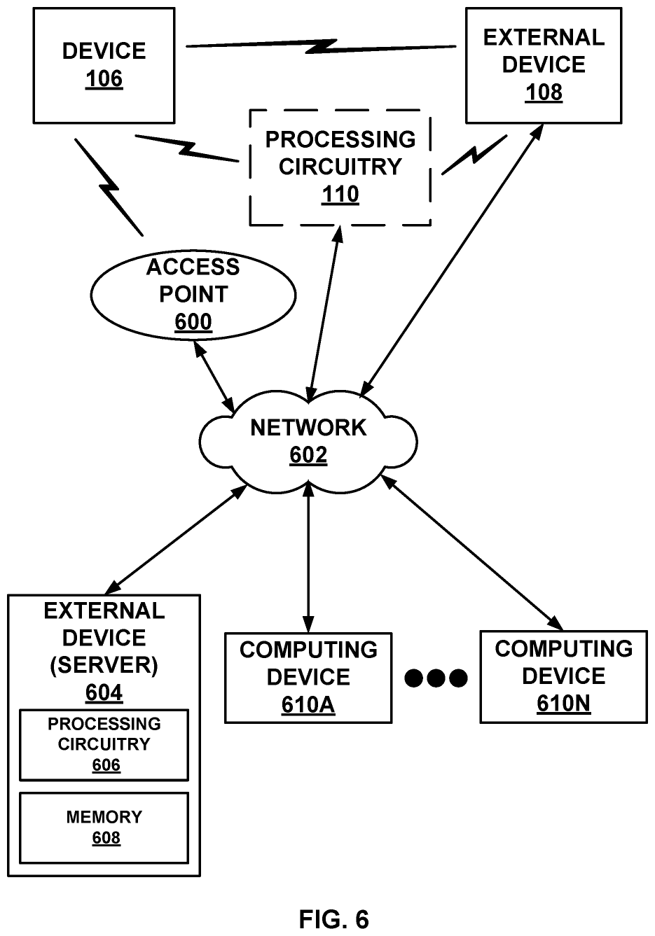

FIG. 6 is a block diagram illustrating an example system that includes an access point 600, a network 602, external computing devices, such as a server 604, and one or more other computing devices 610A-610N, which may be coupled to sensor device 106, external device 108, and processing circuitry 110 via network 602, in accordance with one or more techniques described herein. In this example, sensor device 106 may use communication circuitry to communicate with external device 108 via a first wireless connection, and to communication with an access point 600 via a second wireless connection. In the example of FIG. 6, access point 600, external device 108, server 604, and computing devices 610A-610N are interconnected and may communicate with each other through network 602.

Access point 600 may include a device that connects to network 602 via any of a variety of connections, such as telephone dial-up, digital subscriber line (DSL), or cable modem connections. In other examples, access point 600 may be coupled to network 602 through different forms of connections, including wired or wireless connections. In some examples, access point 600 may be a user device, such as a tablet or smartphone, that may be co-located with the patient. As discussed above, sensor device 106 may be configured to transmit data, such as signals, parameter values determined from signals, or condition/classification indications, to external device 108. In addition, access point 600 may interrogate sensor device 106, such as periodically or in response to a command from the patient or network 602, in order to retrieve such data from sensor device 106, or other operational or patient data from sensor device 106.

Access point 600 may then communicate the retrieved data to server 604 via network 602.

In some cases, server 604 may be configured to provide a secure storage site for data that has been collected from sensor device 106, and/or external device 108. In some cases, server 604 may assemble data in web pages or other documents for viewing by trained professionals, such as clinicians, via computing devices 610A-610N. One or more aspects of the illustrated system of FIG. 6 may be implemented with general network technology and functionality, which may be similar to that provided by the Medtronic CareLink® Network developed by Medtronic plc, of Dublin, Ireland.

Server 604 may include processing circuitry 606. Processing circuitry 606 may include fixed function circuitry and/or programmable processing circuitry. Processing circuitry 606 may include any one or more of a microprocessor, a controller, a DSP, an ASIC, an FPGA, or equivalent discrete or analog logic circuitry. In some examples, processing circuitry 606 may include multiple components, such as any combination of one or more microprocessors, one or more controllers, one or more DSPs, one or more ASICs, or one or more FPGAs, as well as other discrete or integrated logic circuitry. The functions attributed to processing circuitry 606 herein may be embodied as software, firmware, hardware or any combination thereof. In some examples, processing circuitry 606 may perform one or more techniques described herein based on sensed signals and/or parameter values received from sensor device 106. For example, processing circuitry may perform one or more of the techniques described herein to detect, predict, and/or classify one or more patient conditions. In some examples, processing circuitry 110 pay include some or all of processing circuitry 606.

Server 604 may include memory 608. Memory 608 includes computer-readable instructions that, when executed by processing circuitry 606, cause server 604 and processing circuitry 606 to perform various functions attributed to server 604 and processing circuitry 606 herein. Memory 608 may include any volatile, non-volatile, magnetic, optical, or electrical media, such as RAM, ROM, NVRAM, EEPROM, flash memory, or any other digital media.

In some examples, one or more of computing devices 610A-610N (e.g., device 610A) may be a tablet or other smart device located with a clinician, by which the clinician may program, receive alerts from, and/or interrogate sensor device 106. For example, the clinician may access data corresponding to any one or combination of sensed physiological signals, parameters, or indications of detected, predicted, or classified conditions collected by sensor device 106. In some examples, the clinician may enter instructions for a medical intervention for patient 102 into an app in device 610A, such as based on a status of a patient condition determined by sensor device 106, external device 108, processing circuitry 110, or any combination thereof, or based on other patient data known to the clinician. Device 610A then may transmit the instructions for medical intervention to another of computing devices 610A-610N (e.g., device 610B or external device 108) located with patient 102 or a caregiver of patient 102. For example, such instructions for medical intervention may include an instruction to change a drug dosage, timing, or selection, to schedule a visit with the clinician, or to seek medical attention. In further examples, device 610B may generate an alert to patient 102 based on a status of a medical condition of patient 102 determined by sensor device 106, which may enable patient 102 proactively to seek medical attention prior to receiving instructions for a medical intervention. In this manner, patient 102 may be empowered to take action, as needed, to address his or her medical status, which may help improve clinical outcomes for patient 102.

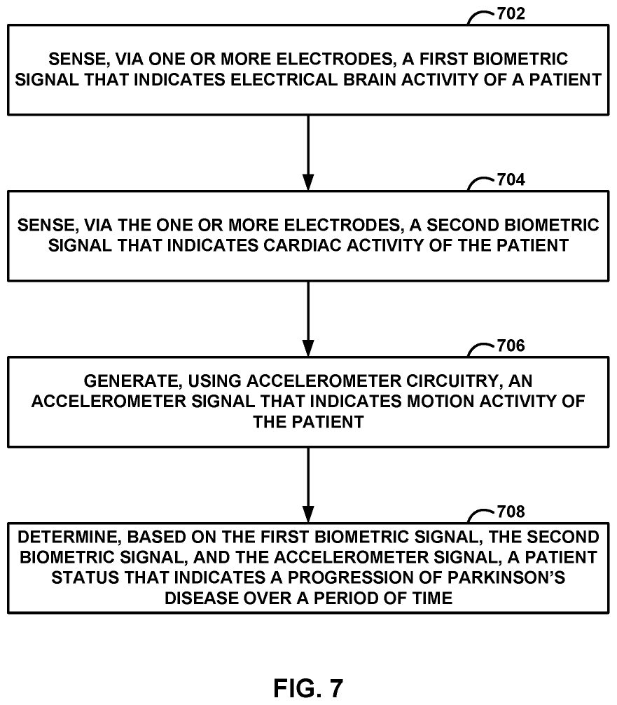

FIG. 7 is a flow diagram illustrating an example operation for determining a patient status that indicates a progression of Parkinson's disease, in accordance with one or more techniques of this disclosure. The example operation is described with respect to sensor device 106, external device 108, and processing circuitry 110 of FIGS. 1A-1B, and components thereof. However, the techniques of FIG. 7 may be performed by different components of sensor device 106, external device 108, and processing circuitry 110, or by additional or alternative medical device systems.

Sensor device 106 may sense, via one or more electrodes, a first biometric signal that indicates electrical brain activity of patient 102 (702). In some examples, to sense the first biometric signal, the sensor device 106 is configured to sense an electroencephalography (EEG) signal that indicates electrical brain activity of patient 102 in a tissue region of the brain of the patient 102. Processing circuitry 110 and/or circuitry of sensor device 106 may filter the EEG signal to identify a frequency band of the EEG signal, wherein a waveform of the frequency band of the EEG signal is indicative of the patient status that indicates the progression of Parkinson's disease. In some examples, processing circuitry 110 and/or circuitry of sensor device 106 may filter the EEG signal to identify a set of frequency bands.

Sensor device 106 may sense, via the one or more electrodes, a second biometric signal that indicates cardiac activity of the patient (704). The second biometric signal may indicate a level of α-synuclein in a tissue area of the patient. Processing circuitry 110 and/or sensor device 106 may filter, based on the level of α-synuclein in a tissue area of patient 102 proximate to patient 102, the second biometric signal to identify a biomarker signal that indicates the cardiac activity of the patient 102. Sensor device 106 may sense, using accelerometer circuitry (e.g., motion sensors), an accelerometer signal that indicates motion activity of patient 102 (706). For example, the accelerometer signal may indicate an amount of tremor motion activity of the patient 102. Processing circuitry 110 may determine, based on the first biometric signal, the second biometric signal, and the accelerometer signal, a patient status that indicates a progression of Parkinson's disease over a period of time (708).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following are illustrative of the techniques described herein.

Example 1: A medical device system comprising: an implantable medical device comprising one or more electrodes; and accelerometer circuitry, wherein the implantable medical device is configured to be located subcutaneously and proximate to a brain of a patient, and wherein the implantable medical device is configured to: sense, via the one or more electrodes, a first biometric signal that indicates electrical brain activity of the patient; sense, via the one or more electrodes, a second biometric signal that indicates cardiac activity of the patient; and generate, using the accelerometer circuitry, an accelerometer signal that indicates motion activity of the patient; and processing circuitry configured to: determine, based on the first biometric signal, the second biometric signal, and the accelerometer signal, a patient status that indicates a progression of Parkinson's disease over a period of time.

Example 2: The medical device system of example 1, wherein the implantable medical device is configured to be implanted beneath the skin of the patient at a location where a neck of the patient meets a head of the patient, wherein the implantable medical device is configured to be implanted between a neck muscle of the patient and a skull of the patient, and wherein the one or more electrodes are configured to face the skull of the patient.

Example 3: The medical device system of example 1, wherein to sense the first biometric signal, the implantable medical device is configured to: sense an electroencephalography (EEG) signal that indicates the electrical brain activity of the patient in a tissue region of the brain of the patient; and filter the EEG signal to identify a frequency band of the EEG signal, wherein a waveform of the frequency band of the EEG signal is indicative of the patient status that indicates the progression of Parkinson's disease.

Example 4: The medical device system of example 3, wherein the frequency band represents a beta frequency band that comprises a range of frequencies from 13 Hertz (Hz) to 30 Hz.

Example 5: The medical device system of example 3, wherein to determine the patient status, the processing circuitry is configured to; identify a change in a waveform of the frequency band of the EEG signal over a period of time; and determine that the change in the waveform of the frequency band of the EEG signal corresponds to a change in the patient status that indicates the progression of Parkinson's disease.

Example 6: The medical device system of example 3, wherein the frequency band is a first frequency band, wherein the implantable medical device is further configured to filter the EEG signal to identify a second frequency band of the EEG signal, and wherein the processing circuitry is further configured to: identify an amplitude and a phase of the first frequency band of the EEG signal; identify an amplitude and a phase of the second frequency band of the EEG signal; and determine, based on the amplitude and the phase of the first frequency band and the amplitude and the phase of the second frequency band, whether phase amplitude coupling exists between the first frequency band and the second frequency band, wherein phase amplitude coupling between the first frequency band and the second frequency band is indicative of the patient status.

Example 7: The medical device system of example 6, wherein the first frequency band represents a beta frequency band that comprises a range of frequencies from 13 Hz to 30 Hz, and wherein the second frequency band represents a gamma frequency band that comprises a range of frequencies from 30 Hz to 80 Hz.

Example 8: The medical device system of example 1, wherein the second biometric signal indicates a level of α-synuclein in a tissue area of the patient; and wherein the implantable medical device is configured to filter, based on the level of α-synuclein in the tissue area, the second biometric signal to identify a biomarker signal that indicates the cardiac activity of the patient.

Example 9: The medical device system of example 8, wherein the processing circuitry is configured to: identify, based on the biomarker signal that indicates the cardiac activity of the patient, one or more cardiac metrics; and determine the patient status based on the one or more cardiac metrics identified based on the biomarker signal.

Example 10: The medical device system of example 8, wherein the one or more cardiac metrics include heart rate, heart rate variability, and beat-to-beat interval variability.

Example 11: The medical device system of example 1, further comprising an external device configured to: sense, via a levodopa (L-dopa) sensor, a third biometric signal that indicates a level of L-dopa in a tissue area of the patient, wherein the processing circuitry is further configured to determine, based on the first biometric signal, the second biometric signal, the third biometric signal, and the accelerometer signal, the patient status that indicates the progression of Parkinson's disease over the period of time.

Example 12: The medical device system of example 1, wherein the processing circuitry is further configured to: determine, based on the accelerometer signal, an amount of tremor motion activity of the patient; and determine the patient status based on the amount of tremor motion activity of the patient.

Example 13: The medical device system of example 1, wherein the implantable medical device comprises the processing circuitry.

Example 14: The medical device system of example 1, wherein an external device comprises the processing circuitry.

Example 15: The medical device system of example 1, wherein a length of the implantable medical device is within a range from 30 millimeters (mm) to 70 mm, wherein a width of the implantable medical device is within a range from 2.5 mm to 15 mm, and wherein a depth of the implantable medical device is within a range from 2 mm to 9 mm.

Example 16: A method of controlling operation of a medical device system comprising an implantable medical device located subcutaneously and proximate to a brain of a patient, the method comprising sensing, via one or more electrodes of the implantable medical device, a first biometric signal that indicates electrical brain activity of the patient, wherein the implantable medical device is configured to be located subcutaneously and proximate to a brain of a patient;
sensing, via the one or more electrodes, a second biometric signal that indicates cardiac activity of the patient; generating, using accelerometer circuitry of the implantable medical device, an accelerometer signal that indicates motion activity of the patient; and determining, by processing circuitry of the medical device system based on the first biometric signal, the second biometric signal, and the accelerometer signal, a patient status that indicates a progression of Parkinson's disease over a period of time.

Example 17: The method of example 16, wherein sensing the first biometric signal comprises: sensing, via the one or more electrodes, an electroencephalography (EEG) signal that indicates the electrical brain activity of the patient in a tissue region of the brain of the patient; and filtering, via the one or more electrodes, the EEG signal to identify a frequency band of the EEG signal, wherein a waveform of the frequency band of the EEG signal is indicative of the patient status that indicates the progression of Parkinson's disease.

Example 18: The method of example 17, wherein the frequency band represents a beta frequency band that comprises a range of frequencies from 13 Hertz (Hz) to 30 Hz.

Example 19: The method of example 17, wherein determining the patient status comprises: identifying, by the processing circuitry, a change in a waveform of the frequency band of the EEG signal over a period of time; and determining, by the processing circuitry, that the change in the waveform of the frequency band of the EEG signal corresponds to a change in the patient status that indicates the progression of Parkinson's disease.

Example 20: The method of example 17, wherein the frequency band is a first frequency band, and wherein the method further comprises: filtering, by the implantable medical device, the EEG signal to identify a second frequency band of the EEG signal; identifying, by the processing circuitry, an amplitude and a phase of the first frequency band of the EEG signal; identifying, by the processing circuitry, an amplitude and a phase of the second frequency band of the EEG signal; and determining, by the processing circuitry and based on the amplitude and the phase of the first frequency band and the amplitude and the phase of the second frequency band, whether phase amplitude coupling exists between the first frequency band and the second frequency band, wherein phase amplitude coupling between the first frequency band and the second frequency band is indicative of the patient status.

Example 21: The method of example 16, wherein the second biometric signal indicates a level of α-synuclein in a tissue area of the patient, and wherein the method further comprises filtering, by the implantable medical device based on the level of α-synuclein in the tissue area, the second biometric signal to identify a biomarker signal that indicates the cardiac activity of the patient.

Example 22: The method of example 20, further comprising: identifying, by the processing circuitry based on the biomarker signal that indicates the cardiac activity of the patient, one or more cardiac metrics; and determining, by the processing circuitry, the patient status based on the one or more cardiac metrics identified based on the biomarker signal.

Example 23: The method of example 16, further comprising sensing, by an external device via a levodopa (L-dopa) sensor, a third biometric signal that indicates a level of L-dopa in a tissue area of the patient, wherein the method further comprises determining, by the processing circuitry based on the first biometric signal, the second biometric signal, the third biometric signal, and the accelerometer signal, the patient status that indicates the progression of Parkinson's disease over the period of time.

Example 24: The method of example 16, further comprising: determining, by the processing circuitry based on the accelerometer signal, an amount of tremor motion activity of the patient; and determining, by the processing circuitry, the patient status based on the amount of tremor motion activity of the patient.

Example 25: A non-transitory computer-readable medium comprising instructions for causing one or more processors to sense, via one or more electrodes of an implantable medical device, a first biometric signal that indicates electrical brain activity of the patient, wherein the implantable medical device is configured to be located subcutaneously and proximate to a brain of a patient; sense, via the one or more electrodes, a second biometric signal that indicates cardiac activity of the patient; generate, using accelerometer circuitry of the implantable medical device, an accelerometer signal that indicates motion activity of the patient; and determine, based on the first biometric signal, the second biometric signal, and the accelerometer signal, a patient status that indicates a progression of Parkinson's disease over a period of time.

1. A medical device system comprising:
an implantable medical device comprising:
one or more electrodes; and
accelerometer circuitry,
wherein the implantable medical device is configured to be located subcutaneously and proximate to a brain of a patient, and
wherein the implantable medical device is configured to:
sense, via the one or more electrodes, a first biometric signal that indicates electrical brain activity of the patient;
sense, via the one or more electrodes, a second biometric signal that indicates cardiac activity of the patient; and
generate, using the accelerometer circuitry, an accelerometer signal that indicates motion activity of the patient; and
processing circuitry configured to:
determine, based on the first biometric signal, the second biometric signal, and the accelerometer signal, a patient status that indicates a progression of Parkinson's disease over a period of time.

2. The medical device system of claim 1, wherein the implantable medical device is configured to be implanted beneath the skin of the patient at a location where a neck of the patient meets a head of the patient, wherein the implantable medical device is configured to be implanted between a neck muscle of the patient and a skull of the patient, and wherein the one or more electrodes are configured to face the skull of the patient.

3. The medical device system of claim 1, wherein to sense the first biometric signal, the implantable medical device is configured to:
sense an electroencephalography (EEG) signal that indicates the electrical brain activity of the patient in a tissue region of the brain of the patient; and
filter the EEG signal to identify a frequency band of the EEG signal, wherein a waveform of the frequency band of the EEG signal is indicative of the patient status that indicates the progression of Parkinson's disease.

4. The medical device system of claim 3, wherein the frequency band represents a beta frequency band that comprises a range of frequencies from 13 Hertz (Hz) to 30 Hz.

5. The medical device system of any of claim 3, wherein to determine the patient status, the processing circuitry is configured to;
identify a change in a waveform of the frequency band of the EEG signal over a period of time; and
determine that the change in the waveform of the frequency band of the EEG signal corresponds to a change in the patient status that indicates the progression of Parkinson's disease.

6. The medical device system of claim 3, wherein the frequency band is a first frequency band,
wherein the implantable medical device is further configured to filter the EEG signal to identify a second frequency band of the EEG signal, and
wherein the processing circuitry is further configured to:
identify an amplitude and a phase of the first frequency band of the EEG signal;
identify an amplitude and a phase of the second frequency band of the EEG signal; and
determine, based on the amplitude and the phase of the first frequency band and the amplitude and the phase of the second frequency band, whether phase amplitude coupling exists between the first frequency band and the second frequency band, wherein phase amplitude coupling between the first frequency band and the second frequency band is indicative of the patient status.

7. The medical device system of claim 6,
wherein the first frequency band represents a beta frequency band that comprises a range of frequencies from 13 Hz to 30 Hz, and
wherein the second frequency band represents a gamma frequency band that comprises a range of frequencies from 30 Hz to 80 Hz.

8. The medical device system of claim 1,
wherein the second biometric signal indicates a level of α-synuclein in a tissue area of the patient; and
wherein the implantable medical device is configured to filter, based on the level of α-synuclein in the tissue area, the second biometric signal to identify a biomarker signal that indicates the cardiac activity of the patient.

9. The medical device system of claim 8, wherein the processing circuitry is configured to:
identify, based on the biomarker signal that indicates the cardiac activity of the patient, one or more cardiac metrics; and
determine the patient status based on the one or more cardiac metrics identified based on the biomarker signal.

10. The medical device system of claim 8, wherein the one or more cardiac metrics include heart rate, heart rate variability, and beat-to-beat interval variability.

11. The medical device system of claim 1, further comprising an external device configured to:
sense, via a levodopa (L-dopa) sensor, a third biometric signal that indicates a level of L-dopa in a tissue area of the patient,
wherein the processing circuitry is further configured to determine, based on the first biometric signal, the second biometric signal, the third biometric signal, and the accelerometer signal, the patient status that indicates the progression of Parkinson's disease over the period of time.

12. The medical device system of claim 1, wherein the processing circuitry is further configured to:
determine, based on the accelerometer signal, an amount of tremor motion activity of the patient; and
determine the patient status based on the amount of tremor motion activity of the patient.

13. (canceled)

14. A method of controlling operation of a medical device system comprising an implantable medical device located subcutaneously and proximate to a brain of a patient, the method comprising:
sensing, via one or more electrodes of the implantable medical device, a first biometric signal that indicates electrical brain activity of the patient, wherein the implantable medical device is configured to be located subcutaneously and proximate to a brain of a patient;
sensing, via the one or more electrodes, a second biometric signal that indicates cardiac activity of the patient;
generating, using accelerometer circuitry of the implantable medical device, an accelerometer signal that indicates motion activity of the patient; and
determining, by processing circuitry of the medical device system based on the first biometric signal, the second biometric signal, and the accelerometer signal, a patient status that indicates a progression of Parkinson's disease over a period of time.

15. (canceled)

16. The medical device system of claim 1, wherein the processing circuitry is further configured to:
determine, based on the accelerometer signal, an amount of tremor motion activity of the patient; and
determine the patient status based on the amount of tremor motion activity of the patient.

17. The medical device system of claim 1, wherein a length of the implantable medical device is within a range from 30 millimeters (mm) to 70 mm, wherein a width of the implantable medical device is within a range from 2.5 mm to 15 mm, and wherein a depth of the implantable medical device is within a range from 2 mm to 9 mm.

18. The method of claim 14, wherein sensing the first biometric signal comprises:
sensing, via the one or more electrodes, an electroencephalography (EEG) signal that indicates the electrical brain activity of the patient in a tissue region of the brain of the patient; and
filtering, via the one or more electrodes, the EEG signal to identify a frequency band of the EEG signal, wherein a waveform of the frequency band of the EEG signal is indicative of the patient status that indicates the progression of Parkinson's disease.

19. The method of claim 18, wherein the frequency band represents a beta frequency band that comprises a range of frequencies from 13 Hertz (Hz) to 30 Hz.

20. The method of claim 18, wherein the frequency band is a first frequency band, and wherein the method further comprises:
filtering, by the implantable medical device, the EEG signal to identify a second frequency band of the EEG signal;
identifying, by the processing circuitry, an amplitude and a phase of the first frequency band of the EEG signal;
identifying, by the processing circuitry, an amplitude and a phase of the second frequency band of the EEG signal; and
determining, by the processing circuitry and based on the amplitude and the phase of the first frequency band and the amplitude and the phase of the second frequency band, whether phase amplitude coupling exists between the first frequency band and the second frequency band, wherein phase amplitude coupling between the first frequency band and the second frequency band is indicative of the patient status.

21. The method of claim 14, wherein the second biometric signal indicates a level of α-synuclein in a tissue area of the patient, and
wherein the method further comprises filtering, by the implantable medical device based on the level of α-synuclein in the tissue area, the second biometric signal to identify a biomarker signal that indicates the cardiac activity of the patient.

22. The method of claim 14, further comprising
sensing, by an external device via a levodopa (L-dopa) sensor, a third biometric signal that indicates a level of L-dopa in a tissue area of the patient,
wherein the method further comprises determining, by the processing circuitry based on the first biometric signal, the second biometric signal, the third biometric signal, and the accelerometer signal, the patient status that indicates the progression of Parkinson's disease over the period of time.
